SEQUENCE LISTING (18 sequences)

SEQ ID NO: 1 - DNA, 23 bp, Artificial Sequence
  Synthetic
tttgaggtatacaggtatttgtc

SEQ ID NO: 2 - DNA, 39 bp, Artificial Sequence
  Synthetic
ttttgaggtatacaggtatttgtcacctcattagattac

SEQ ID NO: 3 - DNA, 43 bp, Artificial Sequence
  Synthetic
ttttgaggtatacaggtatttgtcgtatacctcattagattac

SEQ ID NO: 4 - DNA, 43 bp, Artificial Sequence; identical to SEQ ID NO: 3
  Synthetic

SEQ ID NO: 5 - DNA, 45 bp, Artificial Sequence
  Synthetic
ttttgaggtatacaggtatttgtcctgtatacctcattagattac

SEQ ID NO: 6 - DNA, 19 bp, Artificial Sequence
  Synthetic
aggtatacaggtatttgtc

SEQ ID NO: 7 - DNA, 45 bp, Artificial Sequence
  Synthetic
aggtatacaggtatttgtcacctcattagattaccattagattac

SEQ ID NO: 8 - DNA, 47 bp, Artificial Sequence
  Synthetic
aggtatacaggtatttgtcatacctcattagattaccattagattac

SEQ ID NO: 9 - DNA, 49 bp, Artificial Sequence
  Synthetic
aggtatacaggtatttgtcgtatacctcattagattaccattagattac

SEQ ID NO: 10 - DNA, 39 bp, Artificial Sequence
  Synthetic
tttttnnnnnnnnggcacacgagatttttctcgtgtgcc

SEQ ID NO: 11 - DNA, 37 bp, Artificial Sequence
  Synthetic
tttttnnnnnnggcacacgagatttttctcgtgtgcc

SEQ ID NO: 12 - DNA, 16 bp, Artificial Sequence
  Synthetic
tttttgtaatctaatg

SEQ ID NO: 13 - DNA, 64 bp, Artificial Sequence
  Synthetic
ttttttacctgtatacctggcacacgagatttttctcgtgtgccaggtatacaggtattttgtc

SEQ ID NO: 14 - DNA, 24 bp, Artificial Sequence
  Synthetic
ttttaggtatacaggtattttgtc

SEQ ID NO: 15 - DNA, 20 bp, Artificial Sequence
  Synthetic
aggtatacaggtattttgtc

SEQ ID NO: 16 - DNA, 25 bp, Artificial Sequence
  Synthetic
ttttgaggtatacaggtattttgtc

SEQ ID NO: 17 - DNA, 20 bp, Artificial Sequence
  Synthetic
aggtatacaggtattttgtc

SEQ ID NO: 18 - DNA, 44 bp, Artificial Sequence
  Synthetic
ttttttacctgtatacctggcacacgagatttttctcgtgtgcc

(19) United States
(12) Patent Application Publication
(10) Pub. No.: US 2005/0106596 A1
(43) Pub. Date: May 19, 2005

(54) Nucleic acid analysis methods conducted in small reaction volumes

(75) Inventors: Zbigniev Skrzypczynski, Verona, WI (US); Joerg Baier, Verona, WI (US); Karl Nichols, Madison, WI (US)

Correspondence Address: Mary Ann D. Brow;MEDLEN & CARROLL, LLP, Suite 350, 101 Howard Street, San Francisco, CA, 94105, US

(73) Assignee: Third Wave Technologies, Inc., Madison, WI (US)

(21) Appl. No.: 10/928,965

(22) Filed: Aug. 27, 2004

Related U.S. Application Data
(60) Provisional application No. 60/498,383, filed on Aug. 27, 2003.

(51) Int. Cl. C12Q 1/68; C12P 19/34; C12M 1/34

(52) U.S. Cl. 435/6; 435/91.2; 435/287.2

(57) ABSTRACT

The present invention relates to reactions conducted on or in solid surface materials, including liquid phase reactions within capillary tubes and arrays of capillary tubes or open end reaction microchambers. A wide array of biomolecular and combinatorial synthesis reactions may be used with the present invention. For example, the present invention provides methods for forming and cleaving nucleic acid cleavage structures on or in solid surface materials.

The present application claims priority to U.S. Provisional Application Ser. No. 60/498,383, filed Aug. 27, 2003, which is incorporated herein by reference.

### FIELD OF THE INVENTION

The present invention relates to reactions conducted on or in solid surface materials, including liquid phase reactions within capillary tubes and arrays of capillary tubes or reaction microchambers. A wide array of biomolecular and combinatorial synthesis reactions may be used with the present invention. For example, the present invention provides methods for forming and cleaving nucleic acid cleavage structures on or in solid surface materials.

### BACKGROUND OF THE INVENTION

There is an increasing demand for sensitive, accurate, cost effective, and easy-to-use systems and methods for analyzing and characterizing nucleic acid molecules. A number of useful nucleic acid characterization methods have been developed. However, as these methods have become used more routinely, it has become clear that improvements in practicality and flexibility are desired. Thus, the art is need of new high-throughput systems and methods for improving nucleic acid characterization methods.

### SUMMARY OF THE INVENTION

The present invention relates to reactions conducted on or in solid surface materials, including liquid phase reactions within capillary tubes and arrays of capillary tubes or open end reaction microchambers. A wide array of biomolecular and combinatorial synthesis reactions may be used with the present invention. For example, the present invention provides methods for forming and cleaving nucleic acid cleavage structures on or in solid surface materials.

In some embodiments, a test sample solution used in the systems and methods of the present invention comprises a target nucleic acid molecule. In particular embodiments, the method detects the presence or absence of a polymorphism in the target nucleic acid molecule. In some preferred embodiments, the reagents necessary for carrying out the detection method are contained in a detection reagent solution. In some preferred embodiments, the detection reagent solution comprises components configured for use with a TAQMAN assay or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, the detection reagent solution comprises INVADER oligonucleotides, and 5′ probe oligonucleotides.

In still other embodiments, the present invention provides methods for carrying out detection reactions in small reaction chambers. In particular embodiments, glass capillaries are used as reaction vessels. In some preferred embodiments, the glass capillaries remain open at one or both ends throughout the reaction. In particularly preferred embodiments, the glass capillaries are treated with a blocking agent or a coating.

In some embodiments, the present invention provides a method for detecting the presence of a target nucleic acid (e.g., DNA, RNA, etc.) in a sample, comprising a) providing a microchamber containing a liquid reaction mixture, the liquid reaction mixture comprising a sample suspected of containing a target nucleic acid, and detection assay reagents comprising at least one oligonucleotide configured to form a cleavage structure with said target nucleic acid, a cleavage means capable of cleaving a cleavage structure, wherein said detection assay reagents are configured to produce a detectable signal upon cleavage of a cleavage structure; b) incubating the microchamber under conditions that permit said cleavage reagents to produce a detectable signal; and c) detecting said detectable signal, thereby detecting said target nucleic acid in said sample. In some embodiments, the microchamber comprises a capillary tube. In some preferred embodiments, the detection assay reagents comprise reagents for cleaving an invasive cleavage structure. In some embodiments, the detection assay reagents comprise a ligase.

The present invention is not limited by the nature of the sample. In some embodiments, the sample is a biological sample. In some preferred embodiments, the sample is a blood sample or is derived from a blood sample (e.g., diluted, purified, etc.).

In some embodiments, the microchamber comprises a capillary tube. In some preferred embodiments, the method employs an array of capillary tubes comprising two or more tubes. In some embodiments, two or more of the plurality of capillary tubes contain detection assay reagents configured to detect different target nucleic acids (in some embodiments, it may be desired to have two more tubes also contain reagents for detecting the same target nucleic acid as one or more of the other tubes).

In some embodiments, one or more capillary tube are provided by i) preparing the capillary tube to contain the detection assay reagents, or the sample suspected of containing the target nucleic acid, in dried form within said capillary tube; and ii) adding the sample suspected of containing a target nucleic acid or the detection assay reagents in liquid form to the capillary tube under conditions such that the liquid reaction mixture is formed. In some embodiments, the capillaries containing the dried reagents are stored or prepared as kits or kit components.

In some embodiments, the surface of solid supports, whether used as surfaces for attaching arrays or as microchambers, contains regions that are locally changed in the chemical or physical properties as desired. For example, in some embodiments, introduction of desired chemical or physical changes is accomplished with photochemical approaches (e.g., UV irradiation) or thermal approaches (e.g., IR irradiation).

### DESCRIPTION OF THE DRAWINGS

FIG. 1A-1C shows data analysis from microarray experiments of the present invention. FIG. 1A shows an Excel graph and the corresponding data for reactions performed in a 96-well plate using the same reagent mixes as those used for TEFLON 1536-grid glass plate. Reactions were incubated for 45 minutes at 63° C. and were read with a CYTOFLUOR instrument. FIG. 1B shows data from reactions performed under Method #1 (dry/wet), wherein the reaction volume shown above each column is equal to the sample volume, performed for 45 or 60 minutes at 63° C., as indicated. Negative control reactions (no target) contained 50 μg of tRNA and positive reactions contained 0.01 pM synthetic target. FIG. 1C shows data from reactions performed under Method #2 (wet/wet), wherein the reaction volume shown above each column is equal to the reagent volume plus the sample volume, performed for 45 minutes at 63° C. Negative control reactions (no target) contained 50 μg of tRNA; positive reactions contained 0.01 pM synthetic target.

FIGS. 2A and 2B show data analysis from microarray experiments of the present invention, involving direct detection of human genomic DNA from the SYNQUAD delivery to a 1536 TEFLON grid. FIG. 2A shows data from reactions performed under Method #2 (wet/wet), wherein the reaction volume shown above each column is equal to the reagent volume plus the sample volume, performed for 2 hours at 63° C. Negative control reactions (no target) contained 50 μg of tRNA; positive hgDNA reactions contained the indicated amounts of heterozygous human genomic target DNA. FIG. 2B shows data from reactions performed under Method #1 (dry/wet) using reagents without BSA, wherein the reaction volume shown above each column is equal to the sample volume, performed for 2 hours at 63° C. Negative control reactions (no target) contained 50 μg of tRNA; positive hgDNA reactions contained the indicated amounts of heterozygous human genomic target DNA.

FIG. 3 shows capillary reactions chambers and the course of liquid reagent drying within the capillary in some embodiments of the present invention.

FIG. 4 shows capillary arrays in some embodiments of the present invention (e.g., INVADER Assay Barcode (Capillary) low density array).

FIG. 5 shows capillary arrays in some embodiments of the present invention (e.g., Capillary Invader Assay high density array).

FIG. 6 shows data obtained from a capillary INVADER assay reaction.

FIG. 7 shows data obtained from a capillary INVADER assay reaction.

FIG. 8 shows data obtained from a capillary biplex INVADER assay reaction.

FIG. 9 shows data obtained from polystyrene coated capillary INVADER assay reactions. Reactions contained 10⁵ to 10³ synthetic targets per μl and no blocking reagents were added.

FIG. 10 shows data obtained from PVA coated capillary INVADER assay reactions. Reactions were in Beckman polyvinyl alcohol (n-CHO) coated capillaries and contained 105 copies of synthetic target per ul. No blocking reagents were added. These data showed that positive results were seen without any additional blocking reagents and that the alpha-Innotech system could be used to produce numerical data.

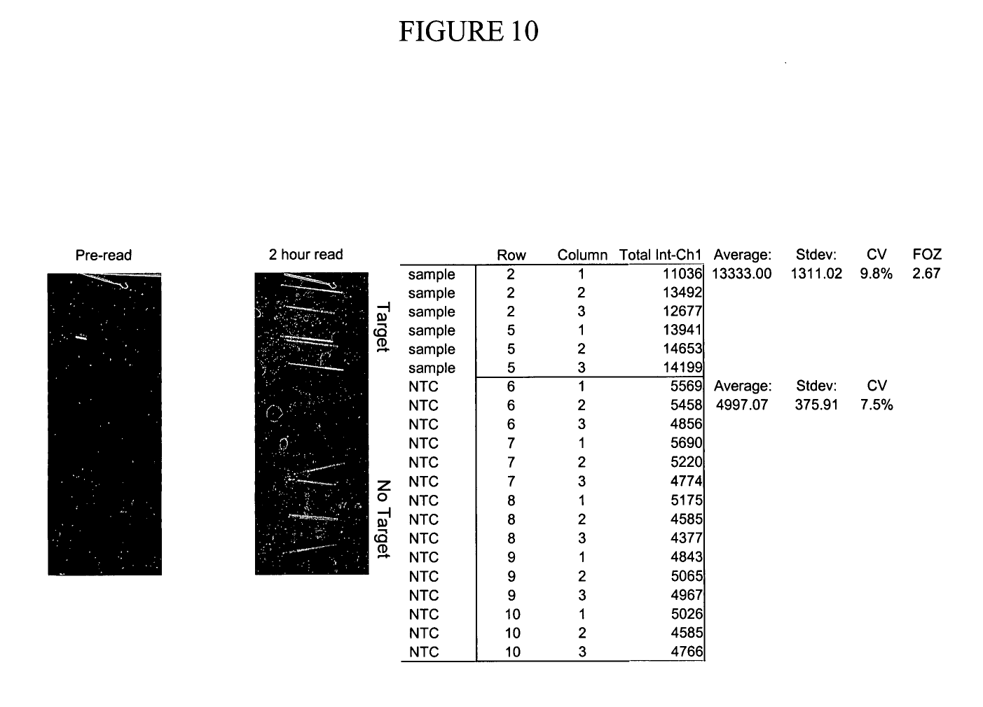

FIG. 11 shows data obtained from array capillary INVADER assay reactions. The assay was run in a 384 well plate for 2 hours and loaded onto TEFLON coated capillaries. Reactions contained 10³ synthetic targets per μl and no blocking reagents were added. The signal was obtained by reading from the top.

FIG. 12 shows a schematic diagram of embodiments involving post-cleavage labeling formats in some embodiments of the present invention.

FIG. 13 shows a schematic diagram of embodiments involving post-cleavage labeling formats in some embodiments of the present invention.

FIG. 14 shows a schematic diagram of embodiments involving post-cleavage labeling formats in some embodiments of the present invention.

FIG. 15 shows a schematic diagram of a configuration for testing embodiments involving post-cleavage labeling formats in some embodiments of the present invention, as described in Example 1.

FIG. 16 shows results from experiments involving post-cleavage labeling formats in some embodiments of the present invention, as described in Example 1.

Definitions

To facilitate an understanding of the present invention, a number of terms and phrases are defined below:

As used herein, the terms “solid support,” “solid surface,” “support,” or “surface” refer to any material that provides a solid or semi-solid structure with which another material can be attached or contained. Such materials include smooth supports (e.g., metal, glass, plastic, silicon, and ceramic surfaces) as well as textured and porous materials. Such materials also include, but are not limited to, gels, rubbers, polymers, and other non-rigid materials. Solid supports need not be flat. Supports include any type of shape including spherical shapes (e.g., beads or microspheres) and fibers. Particular examples of solid supports and methods of using these microparticles for INVADER assays are provided in Stevens et al., Nucleic Acids Research, 29(16):E77, 2001; and Stevens et al., Biotechniques, January;34(1):198-203, 2002, both of which are specifically herein incorporated by reference for all purposes. Materials attached to solid support may be attached to any portion of the solid support (e.g., may be attached to an interior portion of a porous solid support material). Preferred embodiments of the present invention have coatings or biological molecules such as nucleic acid molecules and proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both.

As used herein, the terms “bead,” “particle,” and “microsphere” refer to small solid supports that are capable of moving about in a solution (i.e., have dimensions smaller than those of the enclosure in which they reside). In some preferred embodiments, beads are completely or partially spherical or cylindrical. However, beads are not limited to any particular three-dimensional shape.

As used herein, the term “capillary” refers to a thin tube (e.g., cylindrical or other shaped tupe) with openings at either end capable of containing liquid.

As used herein, the term “microchamber” refers to any small volume containment system, e.g., for an assay reaction. In some embodiments a microchamber comprises one or more solid walls to effect containment. In some embodiments a microchamber comprises one or more regions of having a chemical property that acts to contain a reaction mixture, e.g., a region of hydrophobicity that acts to contain an aqueous reaction mixture.

As used herein, the term “microarray” refers to a solid support with a plurality of molecules (e.g., nucleotides, peptides, etc.) bound to or located on or near its surface. Microarrays, for example, are described generally in Schena, “Microarray Biochip Technology,” Eaton Publishing, Natick, Mass., 2000. Additionally, the term “patterned microarrays” refers to microarray substrates with a plurality of molecules non-randomly bound to or located on or near its surface.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand. Complementarity as used herein is not limited to the predominant natural base pairs comprising the A-T, G-C and A-U base pairs. Rather, the term as used herein encompasses alternative, modified and non-natural bases, including but not limited to those that pair with modified or alternative patterns of hydrogen bonding (see, e.g., U.S. Pat. Nos. 5,432,272 and 6,037,120, each incorporated herein by reference, and others described by Kool, Current Opinion in Chemical Biology, 4:602-608 (2000), incorporated herein by reference.

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, and the Tₘ of the formed hybrid. “Hybridization” methods involve the annealing of one nucleic acid to another complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modem biology.

With regard to complementarity, it is important for some diagnostic applications to determine whether the hybridization represents complete or partial complementarity. For example, where it is desired to detect simply the presence or absence of pathogen DNA (such as from a virus, bacterium, fungi, mycoplasma, protozoan) it is only important that the hybridization method ensures hybridization when the relevant sequence is present; conditions can be selected where both partially complementary probes and completely complementary probes will hybridize. Other diagnostic applications, however, may require that the hybridization method distinguish between partial and complete complementarity. It may be of interest to detect genetic polymorphisms. For example, human hemoglobin is composed, in part, of four polypeptide chains. Two of these chains are identical chains of 141 amino acids (alpha chains) and two of these chains are identical chains of 146 amino acids (beta chains). The gene encoding the beta chain is known to exhibit polymorphism. The normal allele encodes a beta chain having glutamic acid at the sixth position. The mutant allele encodes a beta chain having valine at the sixth position. This difference in amino acids has a profound (most profound when the individual is homozygous for the mutant allele) physiological impact known clinically as sickle cell anemia. It is well known that the genetic basis of the amino acid change involves a single base difference between the normal allele DNA sequence and the mutant allele DNA sequence.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine, as well as other available nucleotide and nucleotide analogues. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “Tₘ ” is used in reference to the “melting temperature.” The melting temperature is the temperature at which a population of double-stranded nucleic acid molecules becomes half disassociated into single strands. Several equations for calculating the Tₘ of nucleic acids are well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G. T mismatches in DNA. Biochemistry 36, 10581-94 (1997)) include more sophisticated computations which take structural and environmental, as well as sequence characteristics into account for the calculation of Tₘ.

As used herein the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids that are not completely complementary to one another be hybridized or annealed together.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42° C. when a probe of about 500 nucleotides in length is employed.

The term “gene” refers to a DNA sequence that comprises control and coding sequences necessary for the production of an RNA having a non-coding function (e.g., a ribosomal or transfer RNA) or encoding a polypeptide or a precursor. The RNA or polypeptide can be encoded by a full-length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprising two or more deoxyribonucleotides or ribonucleotides, preferably at least 5 nucleotides, more preferably at least about 10-15 nucleotides and more preferably at least about 15 to 30 or more nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated. An oligonucleotide “primer” may occur naturally, as in a purified restriction digest or may be produced synthetically.

A primer is selected to be “substantially” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

The term “label” as used herein refers to any atom or molecule or particle that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, particle detection, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc.) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection systems known in the art, or combinations thereof.

The term “cleavage structure” as used herein, refers to a structure that is formed by the interaction of at least one probe oligonucleotide and a target nucleic acid, forming a structure comprising a duplex, the resulting structure being cleavable by a cleavage agent, including but not limited to an enzyme. The cleavage structure is a substrate for specific cleavage by the cleavage agent in contrast to a nucleic acid molecule that is a substrate for non-specific cleavage by agents such as phosphodiesterases that cleave nucleic acid molecules without regard to secondary structure (i.e., no formation of a duplexed structure is required).

The term “folded cleavage structure” as used herein, refers to a region of a single-stranded nucleic acid substrate containing secondary structure, the region being cleavable by an enzymatic cleavage agent. The cleavage structure is a substrate for specific cleavage by the cleavage agent in contrast to a nucleic acid molecule that is a substrate for non-specific cleavage by agents such as phosphodiesterases that cleave nucleic acid molecules without regard to secondary structure (i.e., no folding of the substrate is required).

As used herein, the term “folded target” refers to a nucleic acid strand that contains at least one region of secondary structure (i.e., at least one double stranded region and at least one single-stranded region within a single strand of the nucleic acid). A folded target may comprise regions of tertiary structure in addition to regions of secondary structure.

The term “cleavage means” or “cleavage agent” as used herein refers to any agent that is capable of cleaving a cleavage structure, including but not limited to enzymes. “Structure-specific nucleases” or “structure-specific enzymes” are enzymes that recognize specific secondary structures in a nucleic acid molecule and cleave these structures. The cleavage agents of the invention cleave a nucleic acid molecule in response to the formation of cleavage structures; it is not necessary that the cleavage agents cleave the cleavage structure at any particular location within the cleavage structure.

The term “thermostable” when used in reference to an enzyme, such as a 5′ nuclease, indicates that the enzyme is functional or active (i.e., can perform catalysis) at an elevated temperature, i.e., at about 55° C. or higher.

The term “cleavage products” as used herein, refers to products generated by the reaction of a cleavage agent with a cleavage structure (i.e., the treatment of a cleavage structure with a cleavage agent).

The term “target nucleic acid” refers to a region of a nucleic acid to be detected. In some embodiments, target nucleic acids contain a sequence that has at least partial complementarity with at least a probe oligonucleotide and may also have at least partial complementarity with an INVADER oligonucleotide (described below). The target nucleic acid may comprise single- or double-stranded DNA or RNA. In some embodiments, the term is used to refer to an entire molecule, the presence of which is determined by detecting a portion thereof (e.g., determining the presence of an entire gene by detection of a characteristic portion of that gene). In some embodiments, different portions of a single nucleic acid molecule are to be detected, such that a single molecule comprises more than one target nucleic acid.

The term “probe oligonucleotide” refers to an oligonucleotide that interacts with a target nucleic acid to form a detection complex or cleavage structure. When annealed to the target nucleic acid to form a cleavage structure, cleavage occurs within the probe oligonucleotide.

As used herein, the term “signal probe” refers to a probe oligonucleotide containing a detectable moiety. The present invention is not limited by the nature of the detectable moiety.

As used herein, the terms “quencher” and “quencher moiety” refer to a molecule or material that suppresses or diminishes the detectable signal from a detectable moiety when the quencher is in the physical vicinity of the detectable moiety. For example, in some embodiments, quenchers are molecules that suppress the amount of detectable fluorescent signal from an oligonucleotide containing a fluorescent label when the quencher is physically near the fluorescent label.

The term “non-target cleavage product” refers to a product of a cleavage reaction that is not derived from the target nucleic acid. As discussed above, in the methods of the present invention, cleavage of a cleavage structure generally occurs within the probe oligonucleotide. The fragments of the probe oligonucleotide generated by this target nucleic acid-dependent cleavage are “non-target cleavage products.”

The term “INVADER oligonucleotide” refers to an oligonucleotide that hybridizes to a target nucleic acid at a location near the region of hybridization between a probe and the target nucleic acid, wherein the INVADER oligonucleotide comprises a portion (e.g., a chemical moiety, or nucleotide—whether complementary to that target or not) that overlaps with the region of hybridization between the probe and target. In some embodiments, the INVADER oligonucleotide contains sequences at its 3′ end that are substantially the same as sequences located at the 5′ end of a probe oligonucleotide. As used herein, the term “invasive cleavage structure” refers to the structure formed by an INVADER oligonucleotide and a probe when hybridized to the target nucleic acid, as described above. As used herein, “invasive cleavage assay” refers to an assay configured to detect cleavage of an invasive cleavage structure.

The term “substantially single-stranded” when used in reference to a nucleic acid substrate means that the substrate molecule exists primarily as a single strand of nucleic acid in contrast to a double-stranded substrate which exists as two strands of nucleic acid which are held together by inter-strand base pairing interactions.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

The term “liberating” as used herein refers to the release of a nucleic acid fragment from a larger nucleic acid fragment, such as an oligonucleotide, by the action of, for example, a 5′ nuclease such that the released fragment is no longer covalently attached to the remainder of the oligonucleotide.

The term “Kₘ” as used herein refers to the Michaelis-Menten constant for an enzyme and is defined as the concentration of the specific substrate at which a given enzyme yields one-half its maximum velocity in an enzyme catalyzed reaction.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include comprise modified forms of deoxyribonucleotides as well as ribonucleotides.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The term “source of target nucleic acid” refers to any sample that contains nucleic acids (RNA or DNA). Particularly preferred sources of target nucleic acids are biological samples including, but not limited to cell lysates, blood, saliva, cerebral spinal fluid, pleural fluid, milk, lymph, sputum and semen.

An oligonucleotide is said to be present in “excess” relative to another oligonucleotide (or target nucleic acid sequence) if that oligonucleotide is present at a higher molar concentration than the other oligonucleotide (or target nucleic acid sequence). When an oligonucleotide such as a probe oligonucleotide is present in a cleavage reaction in excess relative to the concentration of the complementary target nucleic acid sequence, the reaction may be used to indicate the amount of the target nucleic acid present. Typically, when present in excess, the probe oligonucleotide will be present in at least a 100-fold molar excess; typically at least 1 pmole of each probe oligonucleotide would be used when the target nucleic acid sequence was present at about 10 fmoles or less.

The term “charge-balanced” oligonucleotide refers to an oligonucleotide (the input oligonucleotide in a reaction) that has been modified such that the modified oligonucleotide bears a charge, such that when the modified oligonucleotide is either cleaved (i.e., shortened) or elongated, a resulting product bears a charge different from the input oligonucleotide (the “charge-unbalanced” oligonucleotide) thereby permitting separation of the input and reacted oligonucleotides on the basis of charge. The term “charge-balanced” does not imply that the modified or balanced oligonucleotide has a net neutral charge (although this can be the case). Charge-balancing refers to the design and modification of an oligonucleotide such that a specific reaction product generated from this input oligonucleotide can be separated on the basis of charge from the input oligonucleotide.

The term “net neutral charge” when used in reference to an oligonucleotide, including modified oligonucleotides, indicates that the sum of the charges present (i.e., R—NH₃⁺ groups on thymidines, the N3 nitrogen of cytosine, presence or absence or phosphate groups, etc.) under the desired reaction or separation conditions is essentially zero. An oligonucleotide having a net neutral charge would not migrate in an electrical field.

The term “net positive charge” when used in reference to an oligonucleotide, including modified oligonucleotides, indicates that the sum of the charges present (i.e., R—NH₃⁺ groups on thymidines, the N3 nitrogen of cytosine, presence or absence or phosphate groups, etc.) under the desired reaction conditions is +1 or greater. An oligonucleotide having a net positive charge would migrate toward the negative electrode in an electrical field.

The term “net negative charge” when used in reference to an oligonucleotide, including modified oligonucleotides, indicates that the sum of the charges present (i.e., R—NH₃⁺ groups on thymidines, the N3 nitrogen of cytosine, presence or absence or phosphate groups, etc.) under the desired reaction conditions is −1 or lower. An oligonucleotide having a net negative charge would migrate toward the positive electrode in an electrical field.

The term “polymerization means” or “polymerization agent” refers to any agent capable of facilitating the addition of nucleoside triphosphates to an oligonucleotide. Preferred polymerization means comprise DNA and RNA polymerases.

The term “ligation means” or “ligation agent” refers to any agent capable of facilitating the ligation (i.e., the formation of a phosphodiester bond between a 3′-OH and a 5′ P located at the termini of two strands of nucleic acid). Preferred ligation means comprise DNA ligases and RNA ligases.

The term “reactant” is used herein in its broadest sense. The reactant can comprise, for example, an enzymatic reactant, a chemical reactant or light (e.g., ultraviolet light, particularly short wavelength ultraviolet light is known to break oligonucleotide chains). Any agent capable of reacting with an oligonucleotide to either shorten (i.e., cleave) or elongate the oligonucleotide is encompassed within the term “reactant.”

The term “adduct” is used herein in its broadest sense to indicate any compound or element that can be added to an oligonucleotide. An adduct may be charged (positively or negatively) or may be charge-neutral. An adduct may be added to the oligonucleotide via covalent or non-covalent linkages. Examples of adducts include, but are not limited to, indodicarbocyanine dye amidites, amino-substituted nucleotides, ethidium bromide, ethidium homodimer, (1,3-propanediamino)propidium, (diethylenetriamino)propidium, thiazole orange, (N-N′-tetramethyl-1,3-propanediamino)propyl thiazole orange, (N-N′-tetramethyl-1,2-ethanediamino)propyl thiazole orange, thiazole orange-thiazole orange homodimer (TOTO), thiazole orange-thiazole blue heterodimer (TOTAB), thiazole orange-ethidium heterodimer 1 (TOED1), thiazole orange-ethidium heterodimer 2 (TOED2) and fluorescein-ethidium heterodimer (FED), psoralens, biotin, streptavidin, avidin, dabcyl, fluorescein, etc.

As used herein, the terms “purified” or “substantially purified” refer to molecules, either nucleic acid or amino acid sequences, that are removed from their natural environment, isolated or separated, and are preferably at least 60% free, more preferably 75% free, and most preferably 90% free from other components with which they are naturally associated. A molecule (e.g., a nucleic acid molecule) that is increased in relative amount compared to other molecules (e.g., by amplification) may also be said to be purified. An “isolated polynucleotide” or “isolated oligonucleotide” is therefore a substantially purified polynucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

### DETAILED DESCRIPTION OF THE INVENTION

The present invention relates to reactions conducted on or in solid surface materials, including liquid phase reactions within glass capillary tubes and arrays of glass capillary tubes. A wide array of biomolecular and combinatorial synthesis reactions may be used with the present invention. For example, the present invention provides methods for forming and cleaving nucleic acid cleavage structures on or in solid surface materials.

The compositions and methods of the present invention are useful for generating microarrays. Preferably the microarrays comprise reagents for performing nucleic acid detection assays (e.g. TAQMAN or INVADER assays).

A. Microarrays and Solid Supports

In some embodiments, the present invention provides microarrays. Microarrays may comprise assay reagents and/or targets attached to or located on or near a solid surface (i.e. a microarray spot is formed) such that a detection assay may be performed on the solid surface. In some preferred embodiments, the microarray spots are generated to possess specific and defined chemical and physical characteristics. In other embodiments, the microarray may comprise a plurality of reaction chambers (e.g., capillaries), for conducting detection assays. In some such embodiments, nucleic acids or other detection assay components are attached to the surface of the reaction chamber. In other embodiments, detection assay components are all in the liquid phase or dried down in the reaction chamber.

As used herein, the term “microarray-spot” refers to the discreet area formed on a solid surface, in a layer of non-aqueous liquid in a microwell, or in a reaction chamber containing a population of detection assay reagents. A microarray-spot may be formed, for example, on a solid substrate (e.g. glass, TEFLON) or in a layer of non-aqeous liquid or other material that is on a solid surface, when a reagent sample comprising detection assay reagents is applied to the solid surface (or film on a solid surface) by a transfer means (e.g. pin spotting tool, inkject printer, etc.). In preferred embodiments, the solid substrate (e.g. modified as described below) contains microwells and the microarray-spots are applied in the microwells. In other embodiments, the solid support serves as a platform on which microwells are printed/created and the necessary reagents are introduced to these microwells and the subsequent reaction(s) take place entirely in solution. Creation of a microwell on a solid support may be accomplished in a number of ways, including; surface tension, and etching of hydrophilic pockets (e.g. as described in patent publications assigned to Protogene Corp.). For example, the surface of a support may be coated with a hydrophobic layer, and a chemical component, that etches the hydrophobic layer, is then printed on to the support in small volumes (e.g., to generate local changes in the physical or chemical properties of the hydrophobic layer). The printing results in an array of hydrophilic microwells. An array of printed hydrophobic or hydrophilic towers may be employed to create micorarrays. A surface of a slide may be coated with a hydrophobic layer, and then a solution is printed on the support that creates a hydrophilic layer on top of the hydrophobic surface. The printing results in an array of hydrophilic towers. Mechanical microwells may be created using physical barriers, +/− chemical barriers. For example, microgrids such as gold grids may be immobilized on a support, or microwells may be drilled into the support (e.g. as demonstrated by BML). Also, a microarray may be printed on the support using hydrophilic ink such as TEFLON. Such arrays are commercially available through Precision Lab Products, LLC, Middleton, Wis. In yet another variant, data of customer preferences with respect to the format of the detection assay array are stored on a database used with components of the invention. This information can be used to automatically configure products for a particular customer based upon minimal identification information for a customer, e.g. name, account number or password. In some embodiments, the desired reactions components (e.g., target nucleic acids or detection assay components) are spotted or delivered into wells and then taken up into small reaction chambers such as capillaries. The reaction then occurs within the reaction chamber.

Many types of methods may be used for printing of desired reagents into microarrays (e.g. microarray spots printed into microwells). In some embodiments, a pin tool is used to load the array (e.g. generate a microarray spot) mechanically (see, e.g., Shalon, Genome Methods, 6:639 [1996], herein incorporated by reference). In other embodiments, ink jet technology is used to print oligonucleotides onto a solid surface (e.g., O'Donnelly-Maloney et al., Genetic Analysis:Biomolecular Engineering, 13:151 [1996], herein incorporated by reference) in order to create one or more micorarray spots in a well.

Examples of desired reagents for printing into/onto solid supports (e.g. with microwell arrays) include, but are not limited to, molecular reagents, such as INVADER reaction reagents, designed to perform a nucleic acid detection assay (e.g., an array of SNP detection assays could be printed in the wells); and target nucleic acid, such as human genomic DNA (hgDNA), resulting in an array of different samples. Also, desired reagents may be simultaneously supplied with the etching/coating reagent or printed into/onto the microwells/towers subsequent to the etching process. For arrays created with mechanical barriers the desired reagents are, for example, printed into the resulting wells. In some embodiments, the desired reagents may need to be printed in a solution that sufficiently coats the microwell and creates a hydrophilic, reaction friendly, environment such as a high protein solution (e.g. BSA, non-fat dry milk). In certain embodiments, the desired reagents may also need to be printed in a solution that creates a “coating” over the reagents that immobilizes the reagents, this could be accomplished with the addition of a high molecular weight carbohydrate such as FICOLL or dextran. In some embodiments, the coating is oil.

Application of the target solution to the microarray (or reaction reagents if the target has been printed down or taken up in a reaction chamber) may be accomplished in a number of ways. For example, the solid support may be dipped into a solution containing the target, or by putting the support in a chamber with at least two openings then feeding the target solution into one of the openings and then pulling the solution across the surface with a vacuum or allowing it to flow across the surface via capillary action. Examples of devices useful for performing such methods include, but are not limited to, TECAN—GenePaint system, and AutoGenomics AutoGene System. In yet another embodiment spotters commercially available from Virtek Corp. are used to spot various detection assays onto plates, slides and the like.

In some embodiments, solutions (e.g. reaction reagents or target solutions) are dragged, rolled, or squeegeed across the surface of the support. One type of device useful for this type of application is a framed holder that holds the support. At one end of the holder is a roller/squeegee or something similar that would have a channel for loading of the target solution in front of it. The process of moving the roller/squeegee across the surface applies the target solution to the microwells. At the end opposite end of the holder is a reservoir that would capture the unused target solution (thus allowing for reuse on another array if desired). Behind the roller/squeegee is an evaporation barrier (e.g., mineral oil, optically clear adhesive tape etc.) and it is applied as the roller/squeegee move across the surface.

The application of a target solution to microwell or reaction chamber arrays results in the deposition of the solution at each of the microwell or reaction chamber locations. The chemical and/or mechanical barriers would maintain the integrity of the array and prevent cross-contamination of reagents from element to element. In some preferred embodiments, materials in the microwells or reaction chambers are dried. In some such embodiments, the reagents are rehydrated by the target solution (or detection assay component solution) resulting in an ultra-low volume reaction mix. In some embodiments, the microarray reactions are covered with mineral oil or some other suitable evaporation barrier or humidity chamber to allow high temperature incubation. The signal generated may be detected directly through the applied evaporation barrier using a fluorescence microscope, array reader or standard fluorescence plate reader.

Advantages of the use of a microwell-microarray, for running INVADER assays (e.g. dried down INVADER assay components in each well) include, but are not limited to: the ability to use the INVADER Squared (Biplex) format for a DNA detection assay; sufficient sensitivity to detect hgDNA directly, the ability to use “universal” FRET cassettes; no attachment chemistry needed (which means already existing off the shelf reagents could be used to print the microarrays), no need to fractionate hgDNA to account for surface effect on hybridization, low mass of hgDNA needed to make tens of thousands of calls, low volume need (e.g. a 100 μm microwell would have a volume of 0.28 nl, and at 10⁴ microwells per array a volume of 2.81 μl would fill all wells), a solution of 333 ng/μl hgDNA would result in ˜100 copies per microwell (this is 33× more concentrated than the use of 100 ng hgDNA in a 20 μl reaction), thus 2.8 μl×333 ng/μl=670 ng hgDNA for 10⁴ calls or 0.07 ng per call. It is appreciated that other detection assays can also be presented in this format.

B. Generating and Using Microarray-Spots With Non-Aqueous Liquids

In certain preferred embodiments, the present invention provides methods for generating microarray spots in wells by applying a detection assay reagent solution to a well containing non-aqueous liquid. In other preferred embodiments, the present invention provides methods of contacting a microarray-spot with a test sample solution (e.g. comprising target nucleic acids) by shooting the test sample solution through a layer of non-aqueous liquid covering the microarray spot. In certain embodiments, the solid supports are coated with sol-gel films (described below in more detail).

In some embodiments, the present invention provides methods comprising; a) providing; i) a solid support comprising a well, ii) a non-aqueous liquid, and iii) a detection reagent solution; and b) adding the non-aqueous liquid to the well, and c) adding the detection reagent solution to the well through the non-aqueous liquid under conditions such that at least one microarray-spot is formed in the well. In other embodiments, the methods further comprise step d) contacting the at least one microarray-spot with a test sample solution. In additional embodiments, the contacting comprises propelling the test sample solution through the non-aqueous liquid in the well.

In particular embodiments, the non-aqueous liquid is oil. In other embodiments, the solid support comprises a plurality of wells, and the method is performed with the plurality of wells. In further embodiments, at least two microarray-spots are formed simultaneously (e.g. in at least two of the plurality of wells).

In some embodiments, the test sample solution comprises a target nucleic acid molecule. In preferred embodiments, the target solution comprises less than 800 copies of a target nucleic acid molecule, or less than 400 copies of a target nucleic acid molecule or less than 200 copies of a target nucleic acid molecule. In particular embodiments, the contacting the microarray-spot with the test sample solution identifies the presence or absence of a polymorphism, or other desired particular sequence to be detected, in the target nucleic acid molecule. In some embodiments, wells are coated with a sol-gel coating (e.g. prior to microarray-spot formation).

In other embodiments, the detection reagent solution comprises components configured for use with a detection assay selected from; TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, the detection reagent solution comprises INVADER oligonucleotides, and 5′ probe oligonucleotides.

In additional embodiments, the contacting is performed with a SYNQUAD nanovolume pipetting system, or other fluid transfer system or device. In preferred embodiments, the commercially available CARTESIAN SYNQUAD nanovolume pipetting system is employed. Similar devices may also be employed, including those described in U.S. Pat. Nos. 6,063,339 and 6,258,103, both of which are specifically incorporated by reference, as well as PCT applications: WO0157254; WO0049959; WO0001798; and WO9942804; all of which are specifically incorporated by reference.

In particular embodiments, at least 2 microarray-spots are formed in the well (or at least 3 or 4 or 5 microarray-sports are formed in each well). In multi-well formats, employing multiple microarray-spots multiplies the number of reactions that can be performed on a single solid support (e.g. if 4 microarray-spots are formed in each of the 1536 wells in an a 1536 well plate, then 6144 microarray-spots would be available for performing detection reactions). In further embodiments, the present invention provides a solid support with a well (or wells) formed by the methods described above.

In some embodiments, the present invention provides methods comprising; a) providing; i) a solid support comprising a microarray-spot, ii) a non-aqueous liquid; and iii) a test sample solution; and b) covering the microarray-spot with a layer of the non-aqueous liquid, and c) contacting the microarray-spot with the test sample solution through the layer of non-aqueous liquid. In other embodiments, the test sample solution comprises a target nucleic acid molecule. In further embodiments, the contacting identifies the presence or absence of at least one polymorphism in the target nucleic acid molecule. In preferred embodiments, the test sample solution comprises a target nucleic acid molecule. In preferred embodiments, the target solution comprises less than 800 copies of a target nucleic acid molecule, or less than 400 copies of a target nucleic acid molecule or less than 200 copies of a target nucleic acid molecule.

In certain embodiments, the microarray-spot comprises components configured for use with a detection assay selected from; TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, the microarray-spot comprises INVADER oligonucleotides, and 5′ probe oligonucleotides.

In some embodiments, the solid support comprises a well, and the microarray-spot is located in the well. In certain embodiments, the non-aqueous liquid is oil. In other embodiments, the solid support comprises a plurality of wells, and the method is performed with the plurality of wells. In particular embodiments, at least two microarray-spots are formed simultaneously. In some embodiments, at least 2 microarray-spots are formed in the well (or at least 3 or 4 or 5 microarray-sports are formed in each well). In multi-well formats, employing multiple microarray-spots multiplies the number of reactions that can be performed on a single solid support (e.g. if 4 microarray-spots are formed in each of the 1536 wells in an a 1536 well plate, then 6144 microarray-spots would be available for performing detection reactions; if etched 3072 well plates are used, additional spots may be formed). In further embodiments, the present invention provides a solid support with a well (or wells) formed by the methods described above.

In some embodiments, the contacting comprises propelling the test sample solution through the non-aqueous liquid in the well. In other embodiments, the non-aqueous liquid is mineral oil. In additional embodiments, the non-aqueous liquid is selected from mineral oil, a seed oil, and an oil derived from petroleum.

In additional embodiments, the contacting is performed with a SYNQUAD nanovolume pipetting system, or other fluid transfer system or device. In preferred embodiments, the commercially available CARTESIAN SYNQUAD nanovolume pipetting system is employed. Similar devices may also be employed, including those described in U.S. Pat. Nos. 6,063,339 and 6,258,103, both of which are specifically incorporated by reference, as well as PCT applications: WO0157254; WO0049959; WO0001798; and WO9942804; all of which are specifically incorporated by reference.

In some embodiments, the present invention provides systems comprising; a) a nonvolume pipetting system (e.g., SYNQUAD), and b) a solid support comprising a microarray-spot, wherein the microarray spot is covering with a layer of a non-aqueous liquid. In other embodiments, the system further comprises a test sample solution.

C. Example of Generating and Using Microarray-Spots Through Mineral Oil

This example describes contacting a microarray-spot covered with mineral oil with a test sample (Method #1). This example also describes generating microarray-spots in microwells by printing through a layer of mineral oil, and then contacting this microarray-spot with a test sample through the layer of mineral oil (Method #2).

Method #1

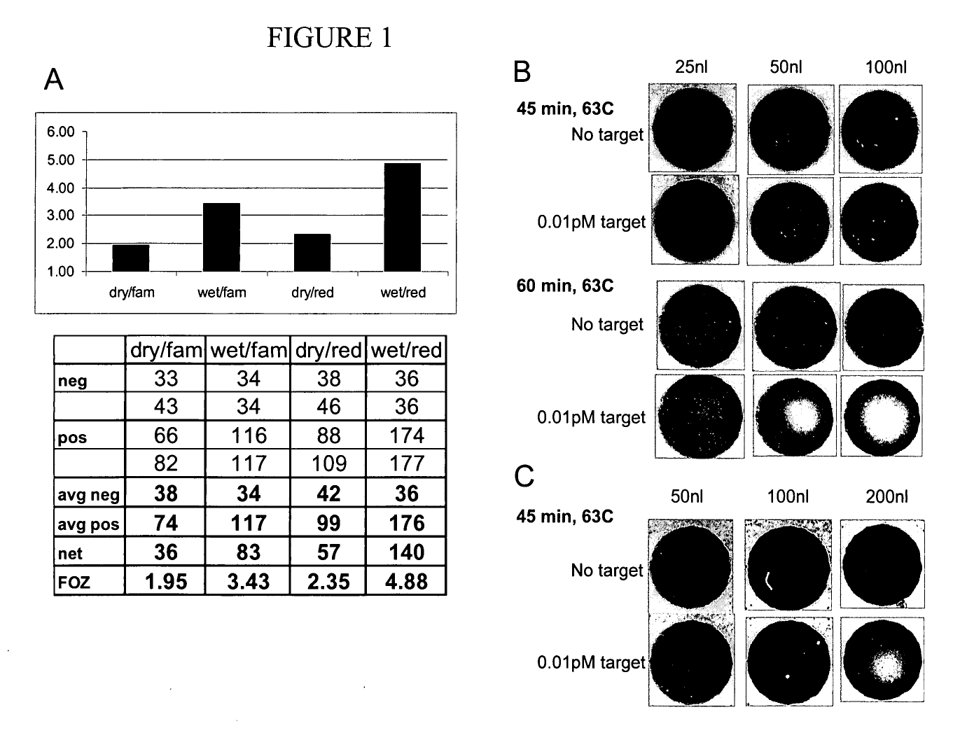

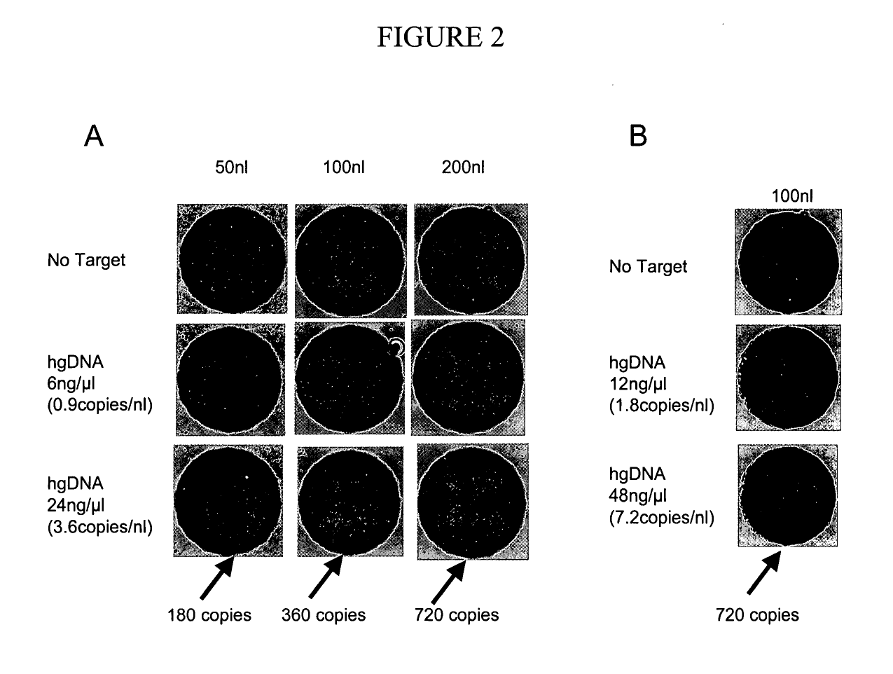

In this method, microarray-spots were generated on a glass solid surface that was divided (by TEFLON printing) into 1536 wells. A CARTESIAN SYNQUAD nanovolume pipetting system was used for fluid transfers. The detection reagent solution employed in this Example was composed of INVADER reaction components, and had the following composition: 10 mM MOPS, 12.5 mM MgCl, 50 ng CLEAVASE XI, 0.1% HPMC 15K cps, 0.2% BSA (Fraction V), 0.5 uM each Primary probe, 0.25 um each FRET cassette, and 0.05 uM INVADER oligonucleotide. The detection assay reagent solution was pipetted into wells using SNYQUAD in volumes of 25, 50, 100, and 200 nl volumes. The solution was then allowed to dry onto glass slides forming microarray spots in the wells. A layer of mineral oil was then applied to the TEFLON 1536 grid glass solid support with CYBIO 384 tip printing head (4 ul per well). Next, SYNQUAD was used to deliver a test sample solution to desired well areas by “shooting” the test sample solution through the mineral oil layer onto the TEFLON 1536 grid glass plate in volumes equal the detection assay reagents printed and dried onto the glass surface (i.e. 25 nl of INVADER assay reagent received 25 nl of test sample). The test sample solution in this method was as follows: negative −50 ng/ul tRNA; positive 0.1 pM each synthetic target). Then the 1536 grid glass plate was incubated in a HERAEUS over at 63 degrees Celsius. Results were analyzed with a fluorescence microscope and CCD camera (Results are presented in FIGS. 1 and 2).

Method #2

In this method, microarray-spots were formed through a layer of mineral oil on the same type of TEFLON 1536 grid glass plate employed in Method #1, and then microarray spots formed were contacted with test sample solution through the mineral oil layer. First, a layer of mineral oil was applied to the TEFLON 1536 grid glass plate with a CYBIO 384 tip printing head (4 ul per well). Next, a detection reagent solution was pipetted into the wells areas using SNYQUAD in volumes of 25, 50, 100 and 200 nl. The detection reagent solution was composed of: 20 mM MOPS, 40 mM MgCl, 110 ng CLEAVASE XI, 5% PEG, 1 uM each primary probe, 0.5 uM each FRET cassette and 0.1 uM INVADER oligonucleotides. Next, a SYNQUAD device was used to deliver test sample solution to desired well areas by shooting the solution through the mineral oil layer on the 1536 grid glass plate in volumes equal to the original detection assay solution. Then, the glass plate was incubated in an HERAEUS over at 63 degrees Celsius. The results were analyzed with a fluorescence microscope and CCD camera. Results are presented in FIGS. 1 and 2.

D. Use of Capillaries as Miniature Reaction Chambers

An alternative type of reaction surface that allows for discrete localization of reaction components is the capillary tube. Tubes may be made of any material. In some preferred embodiments, the tubes are made of glass. In some applications it is desirable to maintain all or some reaction components in a liquid format. The use of capillaries makes it possible to provide liquid reagents in a defined location. Moreover, natural capillary action makes it possible to deliver such liquids to the interior of the capillary simply by contacting one end of the capillary to the source of the liquid, such as a droplet or a well. In some embodiments, it may be desirable to close one or both ends of the capillary once the liquid has been adsorbed, e.g. by heat seal or insertion of a plug (e.g., wax plug). In other cases, it may be preferable to leave one or both ends open to the air.

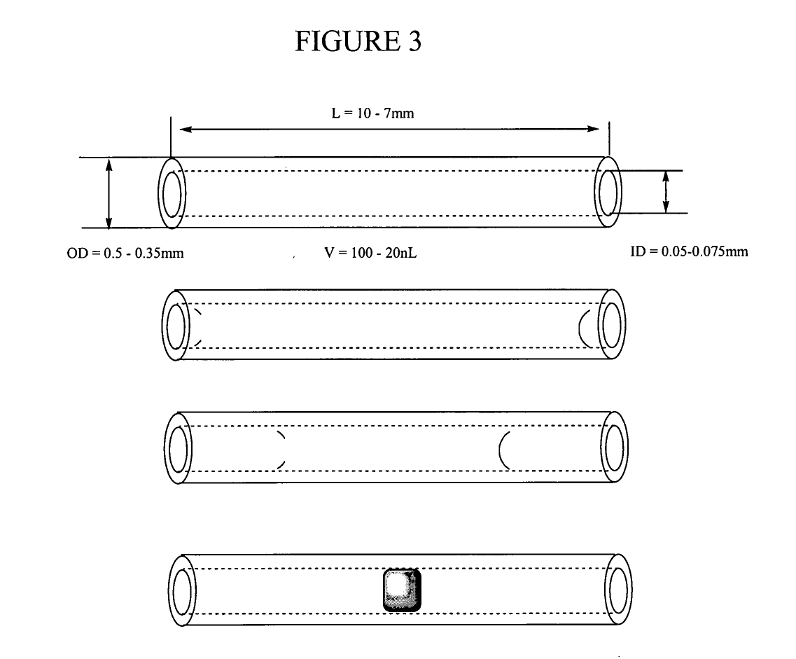

The present invention provides a means of conducting reactions in glass capillaries in the presence of absence of surface modifications, blocking agents, liquid or solid matrices. However, any of the surface modifications or matrices described herein are contemplated for inclusion in the capillary reaction chambers of the present invention. In a preferred embodiment, the capillaries for use in the present invention have dimensions similar to those in FIG. 3. FIG. 3 depicts the course of liquid reagent drying within the capillary. Liquids are adsorbed by capillary action. As drying progresses, the reagents naturally converge toward the center of the capillary. When fluoresently labelled oligonucleotides are included in the liquid, the dried reagents are visible as a tinted spot.

In another preferred embodiment, capillaries for use in the present invention are coated internally with a compound or compounds that yield(s) a uniform, stable, non-reactive substance that does not fluoresce in the range in which fluorescent signal is to be detected. Exemplary compounds possessing these qualities may be covalently or non-covalently attached. Types of non-covalent coatings include, but are not limited to polyamines, polysaccharides, and polymers. Examples of covalent coatings include but are not limited to functional polymers, organosilanes, and metal oxides. Covalent and non-covalent coatings can be hydrophobic or hydrophilic, neutral or charged, synthetic (e.g. C18) or natural (e.g. proteins) high or low molecular weight.

In still another preferred embodiment, capillaries for use in the present invention are coated externally with a compound or compounds that yield(s) a uniform surface with minimal or no fluorescence, possible reflective activity to enhance signal detected within the capilary, and enhanced mechanical stability. Exemplary compounds include, but are not limited to, polyesters, polyamides, TEFLON, polyamide plus a metal such as gold, aluminum, or silver, or metal directly applied to the glass surface.

E. Use of Bundles of Capillaries as Low Density Arrays (LDA) or High Density Arrays (HDA)

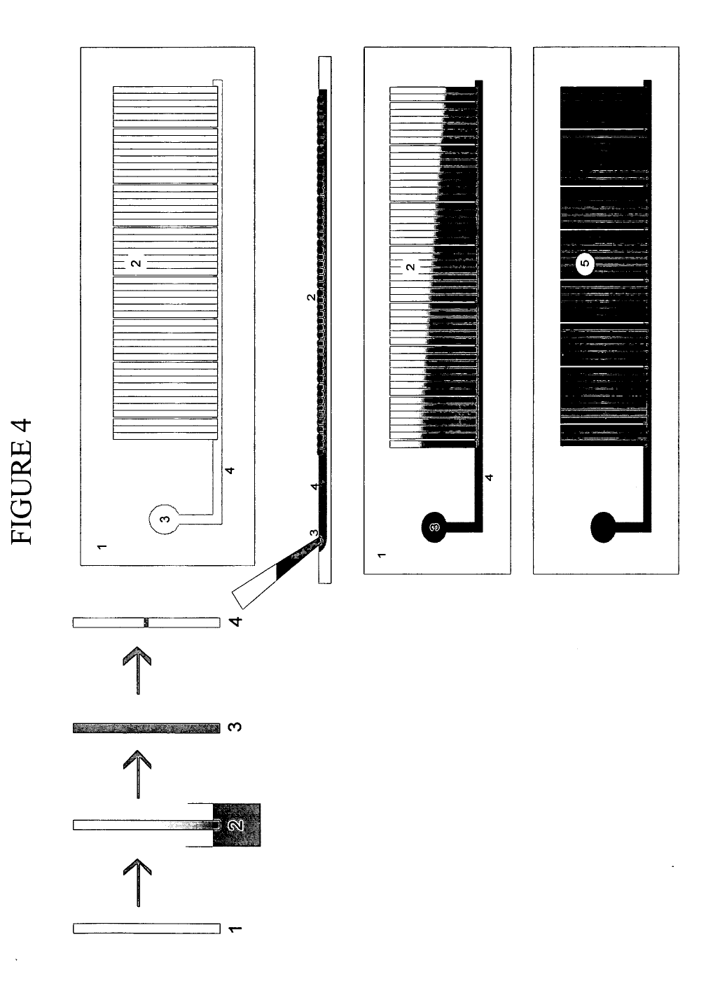

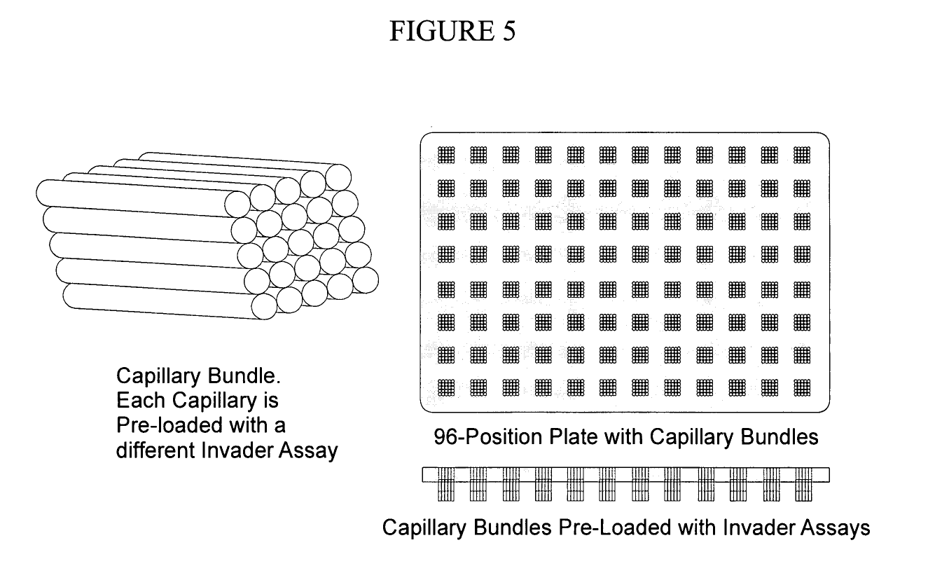

In some embodiments, groups of capillaries, bundled in a row or in a two-dimensional array (alone or in bundles), are assembled to create reaction chamber arrays including, for example, from one to several thousand capillaries. Two examples of such alternative configurations are presented in FIGS. 4 and 5.

The capillary configuration in FIG. 4 depicts one preferred embodiment of a collection of capillaries. The schematic on the left of the figure depicts the stages of capillary loading as follows: (1) shows an empty capillary; (2) shows the capillary contacting a reservoir of liquid which could contain some or all reaction components, sample, or a completed reaction solution; (3) shows a capillary filled with liquid (though the figure depicts the capillary as completely filled, in some embodiments, the capillary may be only partially filled with liquid; and (4) shows a dried spot containing reagents after the capillary is left to air dry or is placed in a drying chamber or dried under vacuum. The top panel on the right depicts a linear bundle of capillaries set in a trough etched into the surface of the support. Suitable supports include glass microscope slides or other similar planar surfaces. The subsequent panels on the right illustrate the addition of a liquid solution to the trough. The capillaries begin to fill purely through the effect of capillary action.

As mentioned above, in some embodiments, it may be desirable to coat the internal capillary surface with functionalized compounds. Examples of such compounds include but are not limited to C-18, diol, PEG, cyclodextrine, PVA, polystyrene, polymethylmetacrylate or other. In other embodiments, it may be desirable to add a blocking reagent to the reaction to minimize interactions between the interior surface of the capillary and the reaction components. Examples of suitable blocking agents, particularly for use in reactions involving nucleic acids and proteins, include dry milk and BSA. The following examples present data obtained from Invader assays carried out in capillaries with or without coatings and with or without blocking reagents. These examples are illustrative and it should be understood that other nucleic analysis technologies may also be used.

### EXAMPLE 1

### INVADER Assays Run in Glass Capillaries

A. Milk Included as Blocking Agent; Reactions Run in Capillaries

Experiments were carried out to determine if INVADER assays could be run directly in glass capillaries. Untreated glass capillaries were handmade from Pasteur pipettes heated over a flame and pulled to elongate and narrow the internal opening, with an outer diameter (OD) of approximately 0.3-0.4 mm and an inside diameter (ID) of approximately 0.2 mm. The length of these capillaries was approximately 10 cm. Capillaries may also be purchased.

Standard INVADER assays (Third Wave Technologies, Madison, Wis.) were utilized, involving a synthetic oligonucleotide target nucleic acid, a primary probe, a FRET cassette, the CLEAVASE XI enzyme (Third Wave Technologies, Madison, Wis.), and 1 ng/μl non-fat dry milk in dH₂O OR dH₂O.

Reaction mixtures were set up either with or without milk added as a blocker in the indicated amounts. In one example, milk was flushed through the capillaries by touching them to a droplet of milk solution (1 ng/μl) and then touching the filled capillaries to paper to extract the liquid but the reaction mixtures adsorbed into these capillaries did not contain milk.

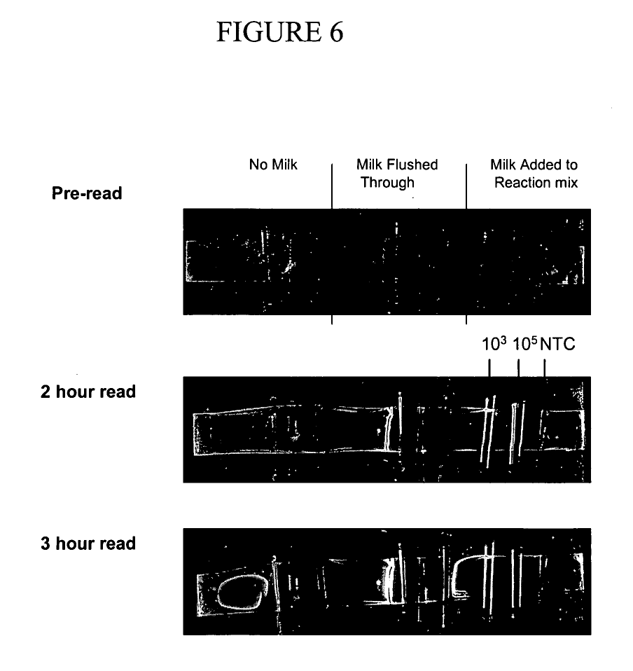

The capillaries were loaded from a drop deposited on a flat surface, via capillary action, with as much of the reaction mixture as would adsorb. Approximately 10-100 nl of the total reaction mixture (from a total of 5 μl) were adsorbed into each capillary. The capillaries were incubated for the indicated read times at 63° C. and then placed in an ALPHAARRAY 7000 fluorescent reader (Alpha Innotech, San Leandro, Calif.). The results are presented in FIG. 6. The capillaries on the left contained reaction mixtures that did not contain milk; the capillaries in the middle were pre-flushed with milk but the reaction mixtures loaded in them did not contain milk; the reaction mixtures on the right contained milk at the indicated concentration. Amounts of synthetic target added to the reactions were the same for each of the test conditions (no milk, milk flushed through, milk added to reaction) and are indicated in the panel on the right, i.e. the left most pair contained 10³ synthetic target molecules, the middle pair, 105, and the right hand pair, no target control (NTC). The results of these experiments indicate that it was possible to discern a difference between the NTC and the experiments containing target nucleic acid when a blocking reagent was included in the reaction.

B. Comparison of Different Blocking Reagents Included in the INVADER Assay

In order to examine the effect of additional blocking reagents as well as coating materials for the internal wall of the capillaries, further comparison experiments were carried out. INVADER reactions were set up as described in Example 1A and included either no blocking agent, BSA (final concentration 0.6%), or milk (final concentration 1 ng/μl). The capillaries used in the experiments were as described in the previous example, except that a comparison was made between uncoated capillaries and capillaries coated with C18. Coating was carried out by immersing handpulled capillaries as described above in a graduated cylinder containing a solution of 7.7 ml ethanol, 5 ml dioxane, 1.3 ml octadecylthiethoxysilane for 20 hours. After 20 hours, the capillaries were removed, washed with water followed by ethanol, vacuum dried and incubated at 80° C. for 1 hour. Finally, the capillaries were cut into pieces 1 cm long.

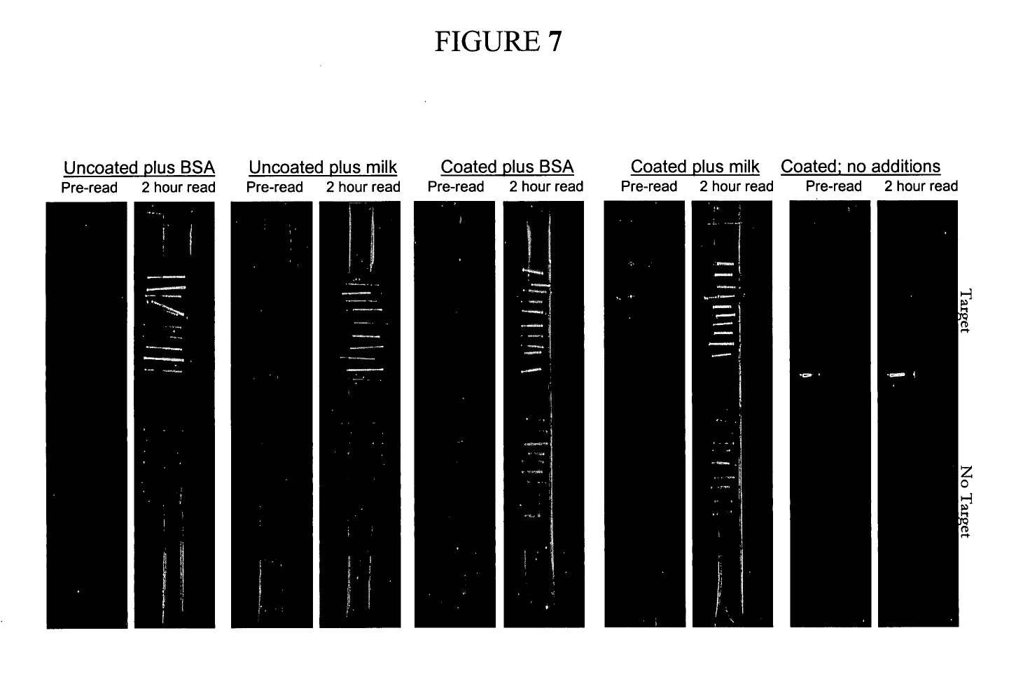

The results of this comparison are presented in FIG. 7 and indicate that in the absence of a blocking agent, even in a C18 coated capillary, little or no signal was generated. By contrast, both blocking agents tested supported the generation of signal in the INVADER assay regardless of whether or not the capillary was coated.

C. Comparison of Effect of Blocking Agents on Biplex INVADER Assay

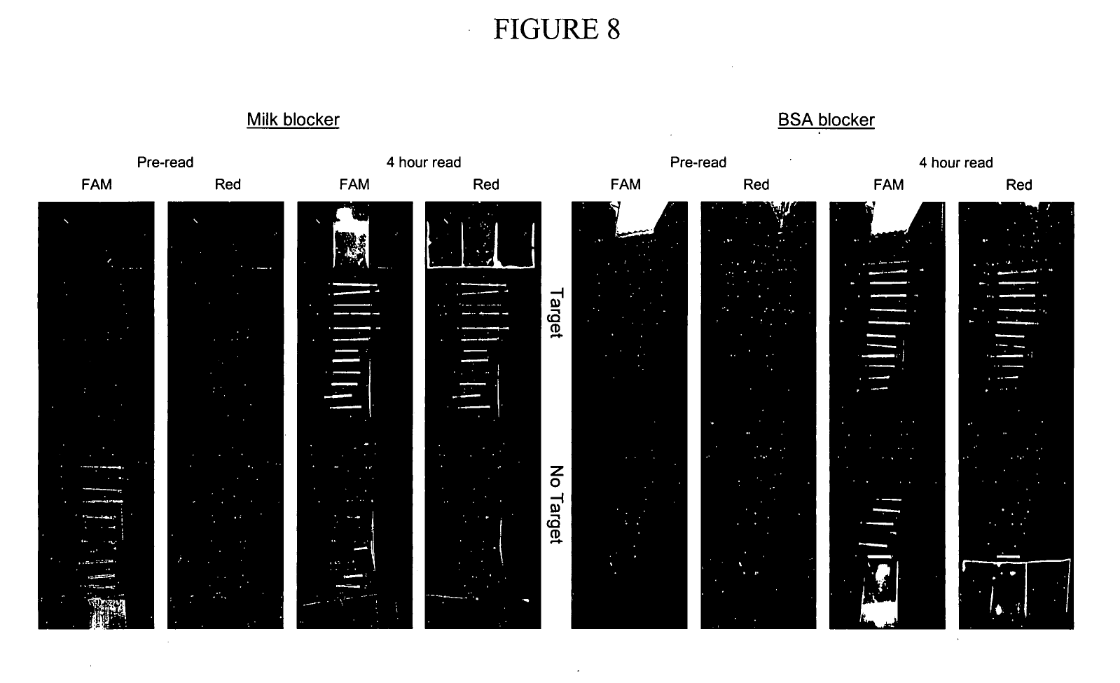

An experiment was carried out in order to evaluate the effect of the two different blocking agents tested in the previous example (1B) on a biplex INVADER assay. MTHFR ASR reagents were used according to the product instructions (Third Wave Technologies, Madison, Wis., part nos. 98-311, 95-312, 97-004). Reactions were run at 63° C. and read as described in the previous examples at the times indicated in FIG. 8. Blocking agents were added as described in the previous experiments. These results indicate that the biplex reactions can be run and detected in capillaries in the presence of either blocking agent.

D. Comparison of the Effects on the INVADER Assay of Different Capillary Coating Materials in the Absence of Blocking Agents

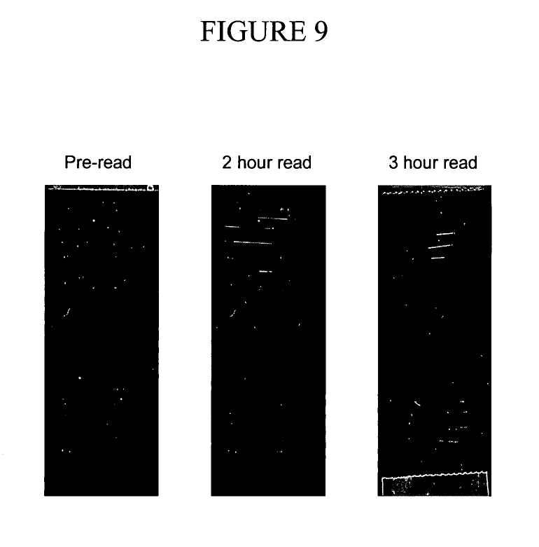

Experiments were carried out to test the effects of additional coatings on INVADER reactions run in capillaries. Two types of capillaries were tested. In one experiment, capillaries coated with C18 were manually coated with polystyrene as follows: 1 cm long capillaries were dipped in a solution of 0.4 g polystyrene pellets (Aldrich, cat. No. 43,010-2, MW=23000, MN=14000) in 10 ml ethyl acetate (Aldrich, HPLC grade) and left overnight at room temperature to dry. The polystyrene plug that formed at the ends was cut off yielding coated capillaries with an effective length of approximately 8 mm. The other type was PVA coated capillary electrophoresis capillaries from Beckman (Fullerton, Calif.; n-CHO capillaries, cat. No. 477601, internal diameter 0.05 mm). The polyester coating on these capillaries was mechanically removed due to undesired fluorescence in the target fluorescent range. INVADER assay reactions were set up as in Examples 1A and B without blocking agent and with the final concentration of target as indicated in the figures and loaded by dipping into the two types of coated capillaries. Reactions were incubated for 2 hours at 63° C. and read as described above. The results are presented in FIGS. 9 (polystyrene) and 10 (PVA) and indicate that both types of coating support signal generation in the INVADER assay even in the absence of additional blocking agents. However, the polystyrene coating appeared to diminish the ability of the capillaries to adsorb liquid through capillary action.

In some embodiments, capillaries are pre-loaded with a subset of reaction components. For example, specific target nucleic acids are deposited in the capillaries. Alternatively, specific oligonucleotides or conjugated proteins are deposited in the capillaries. In some embodiments, it may be desirable to create an addressable orientation for these arrays so that many reactions can be run in parallel and the results readily interpreted by virtue of the position of any given capillary in the array. FIG. 5 depicts one embodiment of a capillary array in which bundles of capillaries are spatially arrayed and then deposited in the wells of a microtiter plate. In a further embodiment, these bundles are tagged in order to fix their orientation in a given well. These bundles can comprise any number of capillaries and can be arrayed on various suitable platforms, including but not limited to 96-well microtiter plates, 384-well microtiter plates, and glass slides. In some preferred embodiments, such capillary bundles are read from the top or bottom to give the view depicted in FIG. 5. Example 2 presents data obtained from loading reaction mixtures in an array of capillaries.

### EXAMPLE 2

### Post Reaction Read in an Array of Capillaries

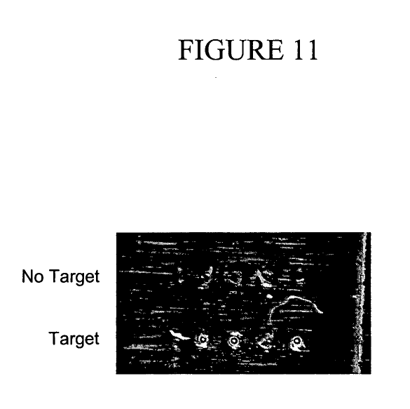

INVADER reactions were carried out as described in Examples 1A and B except that the reactions included 6000 copies of the synthetic DNA target and no blocking agent and were incubated at 63° C. 2 hours in microtiter plate wells and subsequently loaded by capillary action into an array of UV transparent capillaries (TEFLON coated; Polymicro Technologies, Phoenix, Ariz., catalog # 2000140; OD=0.36-0.370 mm; ID=0.102 mm; length=7.9 mm). The array was made by covering a sheet of polyacrylate with aluminum foil and poking holes of approximately the same diameter as the capillaries, and inserting the capillaries into the holes. The array was read as described for the other examples in the ALPHAARRAY7000 reader. The results are presented in FIG. 11 and indicate that this format is suitable for detecting the fluorescent signal generated in the INVADER reaction.

F. Surface Modification, Linker Attachment, and Polymerization Methods

One of the most challenging aspect of the surface modification is the ability to create highly defined areas possessing specific properties different from the surrounding environment, e.g. areas of a high hydrophilicity on the overall hydrophobic surface or areas of highly defined chemical character (reactivity). In most cases this goal is achieved by photochemical modulation of surface properties. However the photolitographic methods that are well developed are both time-consuming and expensive.

The present invention provides an alternate approach of surface modification that provides chemical processes capable of locally changing the character of the solid surface. The chemistry also provides other desired characteristics in that it is fast, efficient, can be non-toxic and can be carried out so as to not leave any unwanted/damaging chemical by-products. The present invention also provides methods for modulating the properties of the surface, as desired.

Any type of solid surface may be employed, including, but not limited to, metal, glass, plastic, silicon, and ceramic surfaces. In certain embodiments, the solid surface comprises microparticles and the methods of using these microparticles for INVADER assays are as described in Stevens et al., Nucleic Acids Research, 29(16):E77, 2001; and Stevens et al., Biotechniques, January;34(1):198-203, 2002, both of which are specifically herein incorporated by reference for all purposes. Additional solid surfaces, and in particular, methods and compositions for performing INVADER assays on solid surfaces, are provided in U.S. application Ser. No. 09/732,622 to Neri et al., which is herein incorporated by reference in its entirety.

In some preferred embodiments, the present invention provides methods for modifying surfaces to generate hydrophobic surfaces that are reactive so as to allow desired molecules to be affixed to the surface—e.g., for the generation of microarrays. In some embodiments, this is accomplished by the production of hydrophobic surfaces using compounds containing disulfide bonds and the conversion of the disulfide bonds into sulfonic acid moieties via oxidation.

In some embodiments, the present invention comprises surface modifications that improve the hydrolytic stability of the bond, e.g. disiloxane, between molecules attached to a surface and the surface itself, e.g. glass. In some embodiments, the improved hydrolytic stability is a result of the hydrophobicity of a portion of the attached molecule. In further embodiments, the attached molecules also comprise a reactive group allowing them to be further modified, e.g. by attaching oligonucleotides.

In some embodiments, the surface modifications can comprise any organic moiety that can undergo a change from hydrophobic to hydrophilic under the influence of the appropriate reagents. Examples of such moieties include, but are not limited to, the following:
—SH to —SO₃
—S₂ to —SO₃
—C≡C— to —COOH
—CH2—X to —CH2—Y, where X is non-polar, e.g. I, Br; and Y is polar (e.g. OH)

Examples of oxidizing agents include, but are not limited to, the following: hydrogen peroxide, nitric acid, sodium periodate, ozone, and DMSO. The use of any particular oxidizing agent is governed by the particular moieties in the reaction. For example, converting —SH to —SO₃ generally may use nitric acid as an oxidizing agent.

Surfaces modified by the methods of the present invention provide arrays with desired surface attached molecules, including but not limited to thiols; disulphides; tricoordinated organophosphorus derivatives; peptides; modified organic polymers such as sugars; DNA; PNA; LNA (for DNA, PNA, LNA, all can be modified).

Embodiments of the present invention are illustrated below with a glass slide as the solid surface. It should be understood that these aspects of the present invention also apply to other surface materials (e.g. gold) and other glass materials (e.g., sol gel).

Initially glass slides were treated with the appropriate, commercially available reagents (purchased from Sigma). However, hydrophobic surfaces, which were produced using those reagents, were not satisfactory from the point of view of their uniformity and stability. For example, glass surfaces are generally not sufficiently homogeneous, and can encounter severe aging problems.

Much better results were generated when methods of the present invention are employed. One such method employs a two-step approach, as diagramed below. In the first step, glass slides are coated with aminosilane. In the second step, amino-modified coated slides are reacted with the desired reagent (L-C(O)—Y; L=leaving group, (e.g., halogen, NHS or other, R=desired organic moiety) capable of reacting with the amino groups of the aminosilane covalently bound to the glass surface.

[image]

For example, in some embodiments, aminopropyl triethoxysilane (R═CH₂CH₂CH₂) was used in the step a) and reactive derivatives of hydrophobic carboxylic acids (oleic acid, stearyl acid, cholesteryl, and perfluoro-aliphatic carboxylic acid) were used in the step b).

Experiments revealed that the procedure described above generated hydrophobic surfaces that were stable and highly uniform across the glass slide. However, it was very difficult to introduce some changes on the created hydrophobic surfaces—i.e. the hydrophobic coating was chemically not sufficiently reactive. For example, it was very difficult to locally change the character of the surface from the hydrophobic to hydrophilic. Therefore the attachment of other materials to those surfaces was weak and the formation of the microarrays of other materials was difficult.

Thus, there was need to develop a better, more flexible coating methodology. In one approach of the present invention, NHS ester of thioctic acid (compound 1) was utilized:

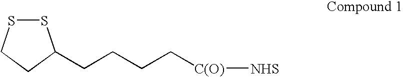

Reacting the NHS ester of the thioctic acid with the aminopropyl triethoxysilane, a new silanizing reagent (compound 2) was generated, capable to introduce on the surface of the glass slide a molecule containing disulfide bond (S—S). This was particularly useful, because of the known lipophilic character of neutral sulfur and because of the relative reactivity of the disulfide bond.

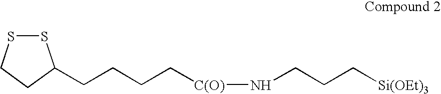

Experiments revealed that the glass surfaces coated with compound 2 were uniform and hydrophobic. Thus, an objective of the designed synthetic strategy was achieved. In the next step, the hydrophobic surfaces coated with the compound 2 were locally treated with 30% solution of the hydrogen peroxide, which is a very aggressive reagent, capable of breaking and oxidizing the S—S bond with the formation of highly hydrophilic sulphonic groups. Oxidation reaction is fast and, as an additional benefit, an excess of the oxidizing reagent (hydrogen peroxide) decomposes to the oxygen and water and evaporates without leaving locally any chemical residues.

It was possible to manually create arrays of hydrophilic spots on the hydrophobic surface by the introduction of small droplets of the hydrogen peroxide using fine glass capillary, microspotter, etc. Many types of methods may be used for printing of desired reagents into microarrays. In some embodiments, a pin tool is used to array the spots mechanically (see, e.g., Shalon, Genome Methods, 6:639 [1996], herein incorporated by reference). In other embodiments, ink jet technology may used to print the droplets of hydrogen peroxide or other suitable reagent onto the hydrophobic surface (e.g., O'Donnelly-Maloney et al., Genetic Analysis:Biomolecular Engineering, 13:151 [1996], herein incorporated by reference). Thus, this coating approach offers significant advantages in the production of hydrophobic arrays, compared to the expensive method of creating of hydrophobic arrays on the gold-coated glass slides. Also, unlike previous methods, the above methods do not require the use of aggressive reagents such as nitric acid or ozone to create hydrophilic spots via oxidation of the SH group.

The above strategy provides the ability to generate a large array of desired compounds on the surface. Thus, the present invention provides a “modular” approach to the modification of the surface properties in the sense that the above chemistry provides dramatic flexibility and control on the identity and position of the molecules to be attached or arrayed on the surface. This idea of synthesis of a large gallery of compounds useful in the modification of the glass surfaces is illustrated in the diagrams below:

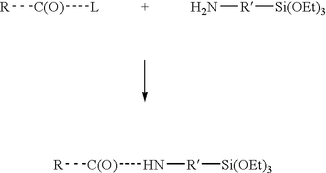

Both groups R and R′ can be selected from a variety of commercially available materials. A large variety of compounds (exemplified in the structure above) capable of derivatizing surfaces can be relatively easy synthesized. Groups R and R′ in those compounds can be selected from aliphatic, aromatic, heterocyclic, or polymeric compounds that will introduce desired structural, chemical or physical properties onto the modified surface.

Those compounds can be used alone or in combination with another silanizing reagents which can, for example, serve as a materials regulating density of the deposition or as additional modifiers that further expands the ability to modulate the properties of the coated glass surface.

One of the most desired property of the silanizing reagents like compound 2, is their ability to interact with the hydroxyl groups of the glass surface and to form relatively stable covalent siloxane bonds (Si—O—Si).

[image]

This bond however, in highly polar medium (water) or at elevated temperatures, can be hydrolytically cleaved. To stabilize the attachment of the coating material to the glass surface, in some embodiments of the present invention, coating the glass slides with organic-inorganic mesoporous sol-gel materials was utilized. The sol-gel method utilizes compounds like compound 2, which in combination with the tetraalkoxysilanes (RO)₄Si and under appropriate reaction conditions (pH) can form hybrid organic-inorganic sol-gel materials.

In some embodiments, porous silicate gels are used in the formation of sol-gel films that find use in coating of glass slides in the production of coated surfaces (e.g., microarrays). The terms “sol-gel glass” and “metal oxide glass” refer to glass material prepared by the sol-gel method and include inorganic material or mixed organic/inorganic material. The materials used to produce the glass can include, but are not limited to, aluminates, aluminosilicates, titanates, ormosils (organically modified silanes), and other metal oxides (See generally, Brinker and Scherer, Sol-Gel Science, Academic Press, San Diego [1995]). In some embodiments, miocroporous inorganic-organic hybrid silicate aerogels are used for the modulation of the physical/chemical properties of the films deposited on the glass surface.

The present invention applies sol-gel materials to surface coating and microarray production, taking advantage of ease of production, very low cost and virtually unlimited scope of synthetic manipulations which can affect the properties (porosity, morphology, optical properties, chemical properties) of the synthesized films. In some embodiments, porous films made out of inorganic-organic silicate hybrids are deposited on the glass surface either by spin coating or by dip coating. Both methods are widely used in the production of new, silicate-based materials. No costly treatments are necessary since the film is deposited in its final form.

The method of sol-gel processing is widely used for making ceramic silica films for the production of microelectronics devices. Those films represent a stable structure which morphology can be easily engineered. When silica based films are formed in the sol-gel process, their structure can be schematically illustrated as a gel-type material formed from the silicon and oxygen bonds, as shown below:

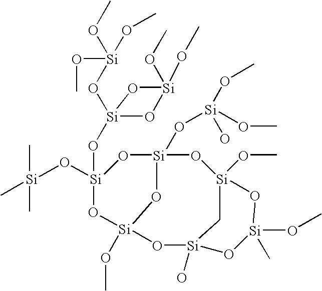

Films composed of such material can be easily deposited on the glass surfaces and modified using a variety of procedures.

One of the most interesting silicate sol-gel films is hybrid inorganic-organic film in which organic molecules are included. A variety of such films made of hybrid aerogels were produces and studied. Their structure is illustrated below:

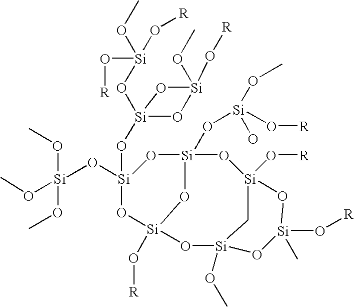

Groups R in the drawing represent an appropriate organic group introduced into the structure via covalent bonds with the silicon atom. The R groups can be identical or different. This increases the flexibility of the design of the properties of the film.

In preferred embodiments of the present invention, the organic groups R have specific chemical reactivity and are an integral part of the structure linking silicon atoms in the film formed in the sol-gel process.

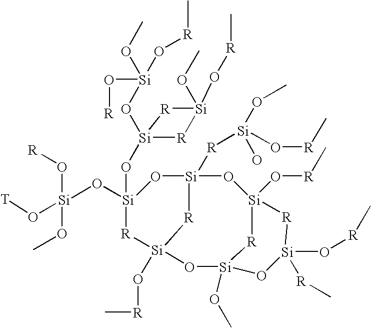

Careful selection of the group R or the use of the different groups R can lead to the formation of films which properties can be modulated. For example, the introduction of compounds containing bisulfide bonds (—S—S—) or sulfhydryl groups (SH) can introduce substantial hydrophobic character and substantial chemical reactivity. The proof of the principle of this concept was demonstrated by the preparation of glass slides coated with the thioctic acid.

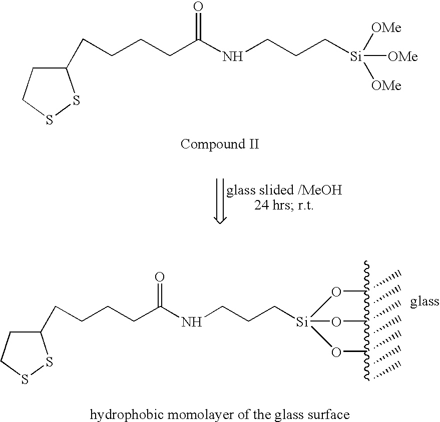

Experiments demonstrated the slides had substantial hydrophobic character and that it is possible to create a highly localized hydrophilic spots by treating the surface with 35% hydrogen peroxide.

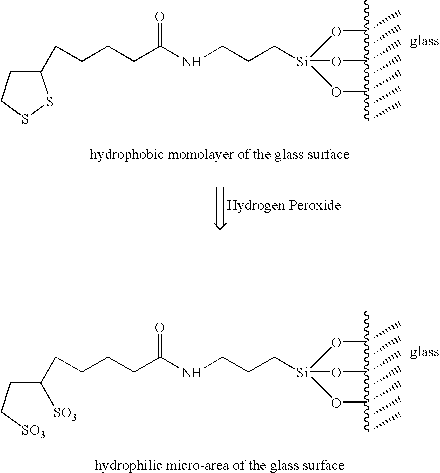

Thus, in some embodiments, the present invention provides microporous hybrid inorganic-organic gels using organic groups R that contain bissulfide groups: —S—S—. In some embodiments, these groups, being part of the mesoporous film, whose thickness can be regulated, can be converted into very polar, hydrophilic sulfonic groups by local application of the hydrogen peroxide.

An advantage of the this approach lies in the fact that the many crucial parameters including film thickness, number of reactive groups and the nature of another organic groups affecting the properties of the aerogel, can be easily regulated. Local application of the appropriate reagent (e.g. hydrogen peroxide) on the surface of the silica film rich in the bisulfide bonds leads to the local disintegration of the structure (local collapse of the structure) and the formation of a micro-well. The whole process can be modulated by the appropriate selection of the organic groups present in the hybrid gel and the reaction conditions.

Preferred embodiments of this method provide:
1. Preparation of glass slides covered with silicate mesophorous films of different thickness
2. Non-covalent modification of such mesoporous films (inorganic and organic modifications)
3. Covalent modification of such mesoporous films (inorganic and organic modifications)
4. Formation of hybrid inorganic-organic mesoporous films of different thickness using different deposition techniques.
5. Formation of mesoporous hybrid silicate films that contain molecules containing bisulfide (—S—S—) or sulfhydryl groups (SH).
6. Formation of mesoporous hybrid inorganic-organic silicate films that contain molecules with any organic groups whose character can be changed in a chemical process leading to the formation of highly localized areas possessing different chemical or physical properties (e.g. hydrophobic-hydrophilic) (e.g., to generate microwells).
7. Depositing of such films hybrid silicate on materials other than glass
8. Preparation of glass slides on which colloidal silica is covalently or non-covalently attached
9. Covalent and non-covalent modification of the colloidal silica and deposition of such colloidal material on solid surfaces like glass, polymer, metal, metalloid.

The present invention also provides approaches that increase the stability of the organic material attached to the glass surface, while also offering multiple points of attachment to the glass surface (“Velcro” approach). It is contemplated that multiple points of attachment improves hydrolytic stability of the coating. An embodiment of this method is diagrammed below.

[image]

Multifunctional materials include, but are not limited to, materials having low molecular weight or from the variety of polymeric materials having the desired chemical of physical properties. Selecting multifunctional polymeric materials rich in hydrophobic groups can offer significant advantage in the stabilization of the attachment of the material to the glass substrate thorough the Si—O bond in highly polar, water based media. While an understanding of the mechanism is not necessary to practice the present invention and the present invention is not limited to any particular mechanism, it is contemplated that the hydrophobic character of the polymeric material protects the points of the attachment of the polymer to the glass substrate by collapsing in the aqueous environment.

In experiments conducted during the development of the present invention, polystyrene-co-maleic anhydride was selected as a substrate for the preparation of hydrophobic multifunctional coating material. Free carboxylic groups of this polymer, dissolved in the organic solvent (dioxane), were first converted into the NHS active esters and subsequently reacted with a) 6-amino-1-hexanol and b) aminopropyltriethoxysilane. The expected material would look like:

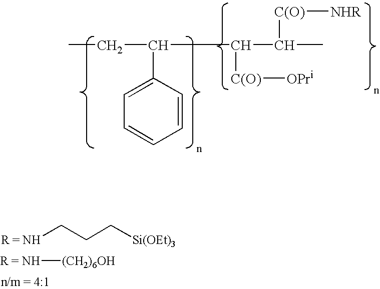

It is contemplated that aminopropyltriethoxysilane moieties attached to the polymeric backbone offer attachment points to the glass substrate and the 6-amino-1-hexanediol introduces free a hydroxyl group that can be a starting point for further chemical manipulations (e.g. chemical DNA synthesis).

In other embodiments, aminoethylaminomethyl phenethyl trimethoxysilane are used to coat surfaces. This material attaches to glass surfaces with good hydrolytic stability (Chen et al., Nanoletters, 2:393 (2000) and Arkles et al., Silica Compounds Register and Review, 5^{th} ed.: United Chemical Technologies; Bristol (1991)). The structure of the material is provided below:

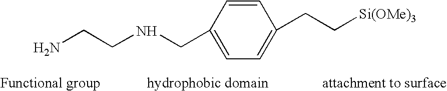

Aminoethylaminomethyl phenethyl trimethoxysilane

This compound, and the one that follows, like all contemplated for this purpose, generally have the following functional domains:
a terminal portion that can attach to a surface, e.g., Si(OR)₃, where R is Me, Et, acetyl;
a hydrophobic linker, which can be as short as C3.
a terminal functional group, e.g., —NH₂, —OH, —COOH, etc.
   An example of another compound having similar properties is shown below:

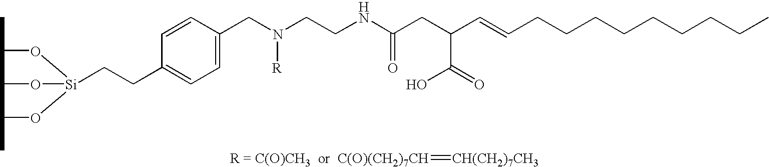

While an understanding of the mechanism is not necessary to practice the present invention and the present invention is not limited to any particular mechanism, it is contemplated that the increased stability of its attachment to the glass surfaces comes from its increased hydrophobic character. Thus, this compound finds use as a substrate for the synthesis of new coating materials prepared according to the “modular approach” described above.

For the design of nucleic acid microarrays, in order to produce new coating reagents offering increased hydrolytic stability and to provide functional groups that serve as a starting point for the chemical oligonucleotide synthesis, this newly identified organosilane was conjugated with the DMT protected NHS ester of 16-hydroxyhexadecanoic acid as illustrated below:

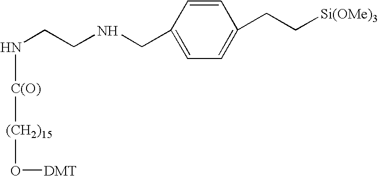

This compound was used in a standard protocol of glass slide modification.

[image]

Experiments demonstrated that oligonucleotides can be synthesized on such slides with excellent stability of the attachment of the synthesized material to the glass surface. The stability of the attachment of organic molecules to glass surfaces thorough the siloxane bond is affected by the hydrophobic nature of the organic groups present in the coating reagent. These chemistries allow a modular approach to the synthesis of new coating reagents, including a great variety of new reagents bearing different structural features (e.g., hydrophilic or hydrophobic character, functional groups, linker length, etc.) that can be synthesized quickly.

The reagent, aminoethylaminomethyl phenethyl trimethoxysilane offers additional features not previously described. As shown in the diagram illustrating the surface of the glass slide coated with the reagent allowing oligonucleotide synthesis, above, the secondary amino group was protected with the trifluoroacetyl group (CF₃C(O)) to eliminate its participation in the process of oligonucleotide synthesis. This structural feature can be exploited as an additional way of introducing desired functionality or functional groups to modulate the properties of the coated surface. [image]
Y=e.g. lipophilic moiety or organic moiety containing crosslinkable groups (like multiple bonds)

Introduction of additional functional group(s) into the coating reagent can be of great utility. As an example, it is contemplated that the introduction of functional groups that undergo polymerization (or cross-linking) under the influence of the appropriate UV wavelengths leads to the formation of very stable and chemically resistant cross-linked polymeric coatings. The stabilization of coated surfaces thorough the UV induced polymerization or cross-linking (curing) of the deposited organic materials an alternative to the use of lipophilic compounds as a way to increase hydrolytic stability of the siloxane bond thorough which material can be attached to the glass surface. Many reagents offering high hydrolytic stability of the molecules attached to the glass surface can be also used as materials that can be exploited in the preparation of ceramic surfaces decorated with polymeric materials (linkers) attached via direct polymerization of the appropriate monomeric units.

In some embodiments, attachment of the long (e.g. PEG based) linkers (MW ˜1100 and 3400) to the glass surface thorough hydrolytically stabilized siloxane bond is prepared. As noted above, such linkers generally comprise a terminal portion that can attach to a surface, a hydrophobic linker, and a terminal functional group. Moieties providing these functions are described above.

Method of Surface Modification Via Direct Growth of the Polymer

The present invention further provides methods and compositions for the chemical modification of solid surfaces useful in the processes of the immobilization of biomaterials. Method finds use, for example, in a process of polymerization of the monomeric units leading to the formation of long linear polymeric structures attached to the solid surface from one end and equipped with the reactive functional group at the other end.

[image]

Polymerization of monomeric blocks may include any kind of polymerization process, i.e., cationic polymerization, anionic polymerization or free radical polymerization. Those processes can be regulated to allow formation of polymers within a relatively narrow range of molecular weight. (e.g., as in ATRP polymerization).

In some preferred embodiments, the method provides solid surfaces densely coated with the long polymeric linkers terminated with functional groups useful in the protocols of immobilization of biomolecules onto the solid surfaces. Depending on the specific synthetic goal and the predicted use of the modified surface, a variety of materials may serve as a substrate for the modification (e.g. modified and unmodified glass surfaces, modified metal surfaces, polymeric surfaces, etc.). Such surface with the polymeric linkers attached to it can serve as a convenient substrate for the chemical synthesis of the DNA probes that would be attached to the solid surface via long polymeric linker. This in turn would eliminate the necessity of multiple couplings prior the synthesis of the oligonucleotide probe (which negatively affects the yield of the final material) or the necessity of the pre-modification of the surface thorough the attachment of the desired polymeric material.

During the development of the present invention, it was found that a recently discovered polymerization process, called Atom Transfer Radical Polymerization [reviewed in Coessens, V. et al., Prog. Polym. Sci. 26: 337-377 (2001)] which allows for the controlled growth of linear polymeric chains, molecular brushes, dendrimers, molecular stars, and thermo-responsive polymers can be used with biomolecules and can be used in the preparation of surfaces decorated with linear polymeric chains. Thus, the present invention provides applications of ATRP in the generation of coated surfaces and microarrays.

The following chemistry finds use with ATRP on solid surfaces to which polymeric linkers will be attached using ATRP process.

[image]

ATRP permits changes in the chemical composition of the polymeric chain throughout its length. For example, the portion of the polymeric chain most proximal to the surface attachment may comprise monomeric units of a first type (e.g., having hydrophobic properties), while more distant portions may comprise monomeric units of a second type (e.g., having hydrophilic properties). One exemplary embodiment is shown below:

[image]

It is contemplated that in water (or water base buffers) environment, such arrangement efficiently protect the point of the attachment the polymer to the glass surface due to the collapsing of the hydrophobic portion of the polymeric chain, as illustrated below.

[image]

Similarly, solid surfaces can be decorated with one or more other polymeric structures generated by the ATRP, including, but not limited to, polymeric brushes, dendrimers, or polymeric mushrooms. The structure of the attached polymeric materials may be homogenous or heterogeneous as desired to limit or expand the scope of their properties and applications.

Using ATRP, a surface can be coated with beads or other attachments having a specific radius creating reactive sites of various densities. Polymeric moieties with multiple reaction sites can be used to attach oligos with varying densities. Similarly, polymers can be used to increases distance from slide surface, minimizing surface-oligo interactions Polymeric structure can be charged to enhance hybridization rates and can be modulated by temperature or chemical means. In addition, mixed polymers can be generated which span a gradient ranging from, for example, fully hydrophobic monomers near the attachment surface to hydrophilic monomers at the free terminus.

ATRP provides a useful method for a variety of biological applications. For example, ATRP may be used to control the density of molecules on a surface. In one such embodiments, ATRP is use to produce beads that are affixed to a molecule of interest (e.g., a nucleic acid molecule). A surface is then coated with the beads (or other attachments) having a specific radius creating reactive sites of desired densities. More dense arrays are produced by selecting smaller radii. Polymeric moieties generated by ATRP, with multiple reaction sites, can be used to attach desired molecule with varying densities. Similarly, ATRP polymers can be used to increases the distance of the desired molecule from the surface, minimizing interactions between the desired molecule and the surface and/or positioning the desired molecule in physical space for optimal functionality.

ATRP also finds use in a number of other biotechnology applications. Any application that benefits from the design of a chemical linker with one or more desired functional properties can accomplished using linkers designed and generated by ATRP. For example, chemical linkers can be attached to nucleic acid molecules or protein molecules to provide functional groups that assist in the purification, identification, isolation, analysis or use of the molecules (e.g., by providing chemical groups that impart one or more unique properties to the molecules containing the linker, including, but not limited to, charge, solubility, size, reactivity, detectability, stability, etc.). Modifications of nucleic acids and proteins can be made to improve binding to binding partners (e.g., increase ligand-receptor bindings, increased hybridization, etc.), cell permeability and therapeutic benefit for antisense oligonucleotide technologies, and the like.

G. Nucleic Acid Detection Assays

As noted above, the methods and compositions of the present invention (e.g. microarrays with modified surfaces, methods for spotting though non-aqueous liquids, conducting assays in small reaction chambers, etc.) are preferably employed with reagents for performing nucleic acid detection assays. In preferred embodiments, the present invention finds application in the practice of the INVADER assay. The INVADER assay detects hybridization of probes to a target by enzymatic cleavage of specific structures by structure specific enzymes (See, INVADER assays, Third Wave Technologies; See e.g., U.S. Pat. Nos. 5,846,717; 6,090,543; 6,001,567; 5,985,557; 6,090,543; 5,994,069, 6,348,314, 6,692,917, 6,555,387; Lyamichev et al., Nat. Biotech., 17:292 (1999), Hall et al., PNAS, USA, 97:8272 (2000), WO97/27214 and WO98/42873, each of which is herein incorporated by reference in their entirety for all purposes). Additional detection assays are provided below (and additional detail is provided on the INVADER assay) to illustrate exemplary nucleic detection assays that could be used with the methods and compositions of the present invention.

i. PCR Assays

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele. PCR reagents may be employed with the methods and compositions of the present invention, for example, to generate microarrays.

ii. Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type. Fragments length polymorphism assay reagents may be employed with the methods and compositions of the present invention, for example, to generate microarrays.

a. RFLP Assay

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are generally separated by gel electrophoresis and may be visualized by ethidium bromide staining. The length of the fragments is compared to molecular weight markers and fragments generated from wild-type and mutant controls.

b. CFLP Assay

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. In preferred embodiments, one or both strands are labeled. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by denaturing gel electrophoresis) and visualized (e.g., by autoradiography, fluorescence imaging or staining). The length of the fragments is compared to molecular weight markers and fragments generated from wild-type and mutant controls.

iii. Hybridization Assays

In preferred embodiments of the present invention, variant sequences are detected a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below. Hybridization assay reagents may be employed with the methods and compositions of the present invention, for example, to generate microarrays.

a. Direct Detection of Hybridization

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991]). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Enzymatic Detection of Hybridization

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with a fluorescent dye that is quenched by a second dye or other quenching moiety. Upon cleavage, the de-quenched dye-labeled product may be detected using a standard fluorescence plate reader, or an instrument configured to collect fluorescence data during the course of the reaction (i.e., a “real-time” fluorescence detector, such as an ABI 7700 Sequence Detection System, Applied Biosystems, Foster City, Calif.).

The INVADER assay detects specific mutations and SNPs in unamplified genomic DNA. In an embodiment of the INVADER assay used for detecting SNPs in genomic DNA, two oligonucleotides (a primary probe specific either for a SNP/mutation or wild type sequence, and an INVADER oligonucleotide) hybridize in tandem to the genomic DNA to form an overlapping structure. A structure-specific nuclease enzyme recognizes this overlapping structure and cleaves the primary probe. In a secondary reaction, cleaved primary probe combines with a fluorescence-labeled secondary probe to create another overlapping structure that is cleaved by the enzyme. The initial and secondary reactions can run concurrently in the same vessel. Cleavage of the secondary probe is detected by using a fluorescence detector, as described above. The signal of the test sample may be compared to known positive and negative controls. Methods and compositions for performing INVADER assays on solid surfaces are provided in U.S. application Ser. Nos. 09/732,622 and 10/309,584 to Neri et al., as well as U.S. Provisional Application 60/374,642 to Lyamichev, all of which are herein incorporated by reference in their entireties.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of DNA polymerases such as AMPLITAQ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labelled antibody specific for biotin).

iv. Other Detection Assays

Additional detection assays that are produced and utilized using the systems and methods of the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); and sandwich hybridization methods (e.g., U.S. Pat. No. 5,288,609, herein incorporated by reference in its entirety). Reagents from these additional nucleic acid detection assay may be employed with the methods and compositions of the present invention, for example, to generate microarrays.

H. Post-Cleavage Labeling of Reaction Products

In order to avoid high development and production costs of photoactivated phosphoramidites containing dyes and quenchers, it may be desirable in some cases to employ alternative detection schemes comprising post-reaction labeling to detect cleavage. As described below, nucleic acid arrays for the INVADER assay can be generated on solid surface arrays (e.g. those produced by NimbleGen, Madison Wis., and those described in U.S. Pat. No. 6,375,903, specifically incorporated herein for all purposes) and used with the post-cleavage labeling methods described below.

In one embodiment of the solid surface INVADER assay, probe oligonucleotides are provided linked to the surface at their 5′ ends. This format leads to a very simple post-reaction labeling scheme with a universal labeling oligonucleotide directly ligated to the 5′ flap of cleaved probes. Target specific cleavage of the probe would result in the formation of a 3′-OH at the end of the 5′ flap sequence present on the probe. The flap sequence, for example, could be one of four different flap sequences, one for each possible base, that together act as a universal system for downstream label attachment. After the INVADER reaction, the solid surface may be washed under denaturing conditions and then exposed to a solution containing CLEAVASE enzyme (or similar enzymes) and four labeled cassettes complementary to each of the four flap sequences. The 5′-flap from probe oligonucleotide creates an overlapping structure with the complementary cassette that results in the formation of a 5′-phosphate on the label cassette. A ligase enzyme, either added simultaneously or in a sequential step, covalently links the labeling cassette to the cleaved flap. Unligated cassettes are then stringently washed from the solid surface (array), leaving the label attached to only cleaved probes.

In another embodiment of the solid-surface INVADER assay, probe oligonucleotides are linked to the surface via their 3′ ends. This format complicates the application of a universal post-reaction labeling scheme because the portion of the cleaved probe that remains attached to the array surface is target specific and varies from assay to assay.

In one embodiment, outlined in FIG. 3, the probe design includes two supplemental sequences, U and A′, located 3′ of the target specific sequence. The A′ sequence is complementary to a portion “A” of the target specific sequence. Target specific cleavage of the probe results in the removal of the 5′-base, resulting in a probe sequence with a 5′-phosphate (FIG. 3A). After the INVADER reaction, the solid surface (e.g. slide) is washed under denaturing conditions and then incubated at a temperature that allows sequence A to anneal to A′, forming the structure indicated in FIG. 3B. Ligase and a universal labeling oligonucleotide containing a label (e.g. a fluorescent dye), U′, is added to the solution. Annealing of the labeling oligonucleotide U′ to U results in the formation of a nick structure and ligation of the nick structure covalently links a label to the cleaved probe.

This labeling scheme increases probe length by the combined length of the U and A′ sequences. The A and A′ sequences should be carefully designed to ensure stable duplex formation at the labeling step but without interfering with formation of the overlapping substrate in the INVADER reaction.

An alternative embodiment involves a degenerate labeling oligonucleotide such as that shown in FIG. 4. The probe-binding region of this oligonucleotide would include a short degenerate region. In a preferred embodiment, this region would comprise 6-8 bases, with all the bases (e.g. natural bases) equally present at each position. This approach would allow any cleaved probe on the array to be labeled in a single step. Both T4 and T7 ligase can ligate contiguous hexamers, suggesting that these duplexes should be sufficiently long (Kaczorowski and Szybalski (1994), Anal Biochem, 221:127-35.; Dunn, et al. (1995), Anal Biochem, 228: 91-100). With each position being degenerate results in a degeneracy factor of 4ⁿ, where n is the number of bases made degenerate, such that a 6 base region would result in a degeneracy factor of 4⁶=4,096. Therefore, a 4 μM mixture of labeling oligonucleotides would contain, for example, approximately 1 nM of each unique sequence (e.g., well within the range of the sensitivity of many fluorescence detection instruments). In the event that this format leads to substantial non-specific background, an additional ligation step with an unlabeled degenerate oligonucleotide before the INVADER reaction can be used to block the non-specific sites.

A further embodiment involves a target specific labeling oligonucleotide to result in a non-universal labeling format. This approach is exemplified in FIG. 5. Instead of using degenerate oligonucleotide mixtures, specific labeling oligonucleotides are created for each target sequence.

### EXAMPLE 1

### Labeling of Cleaved Probes Linked to the Surface Via 3′ Attachment

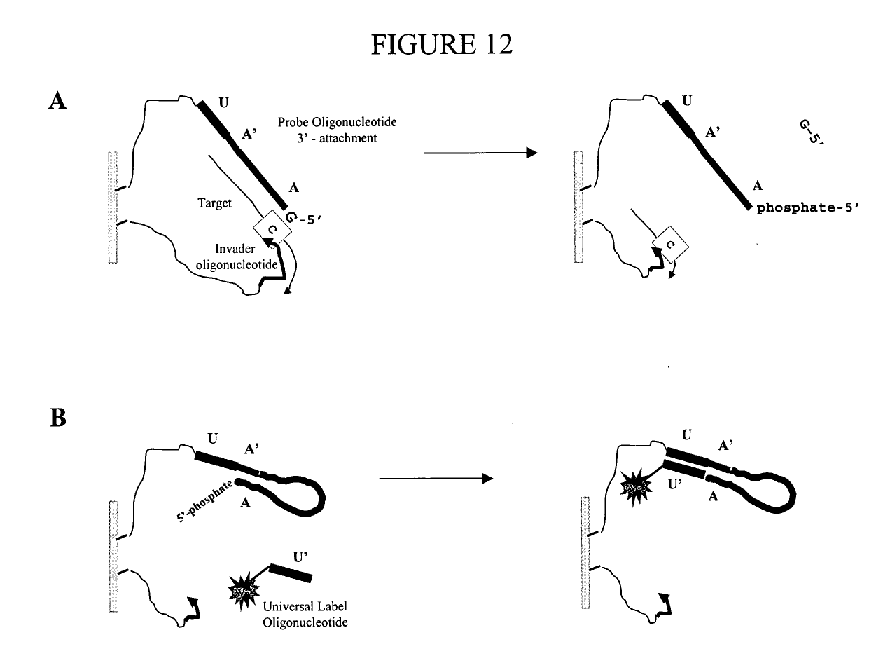

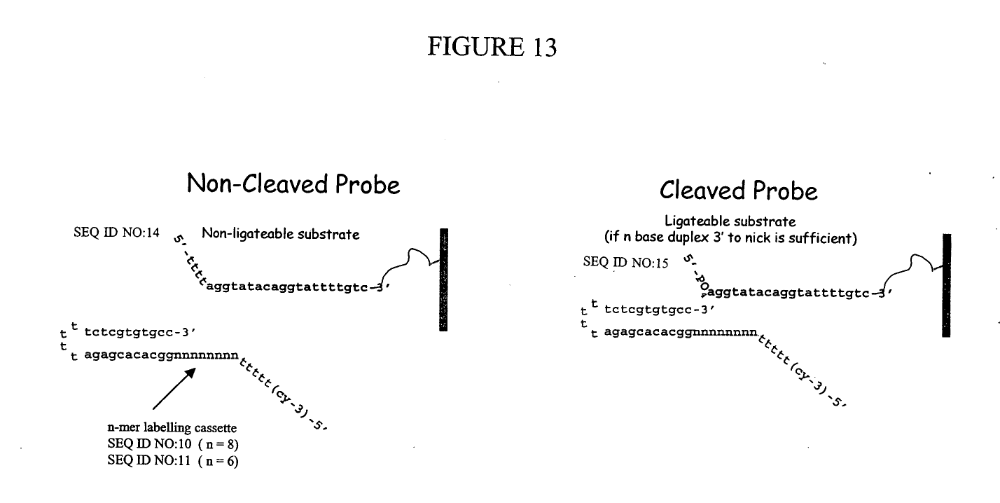

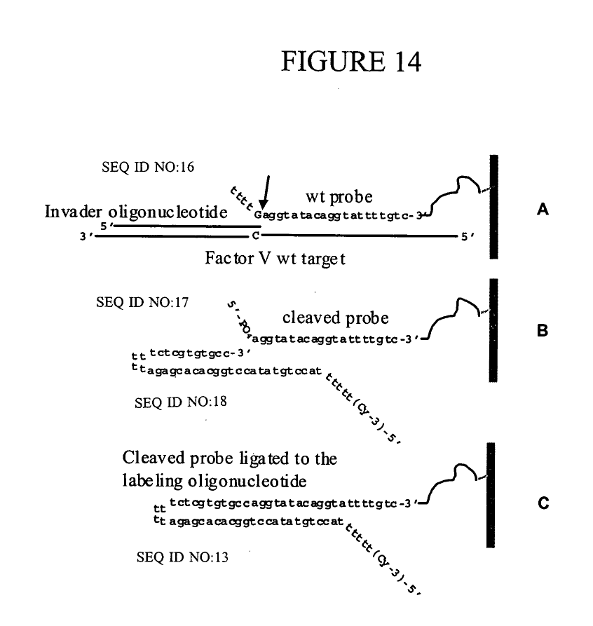

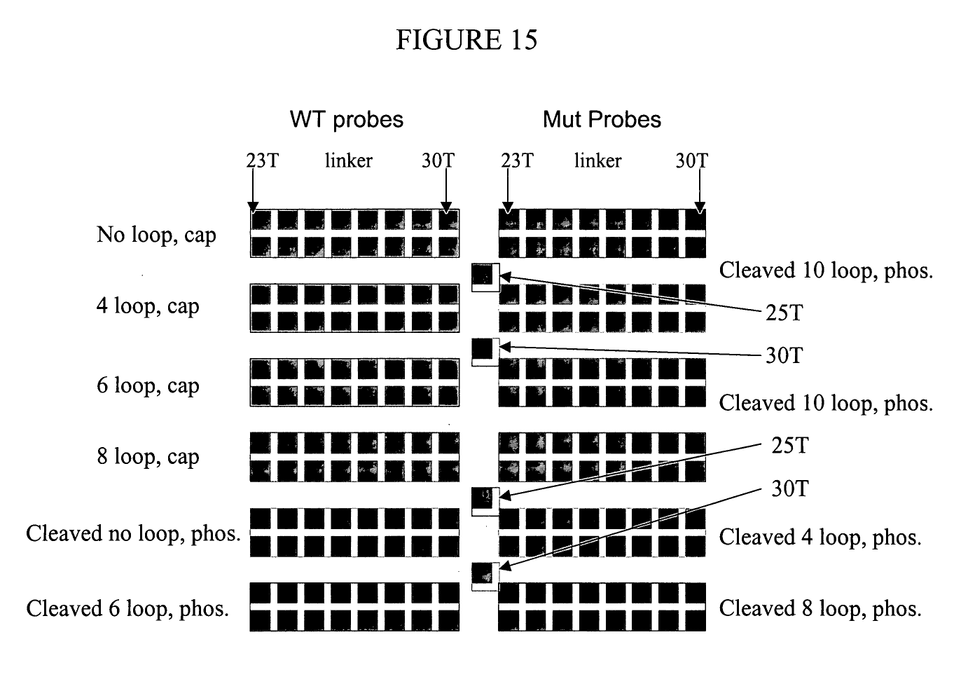

This example compares the different post-cleavage labeling formats shown in FIGS. 12-14. Surfaces were prepared with oligonucleotides on NimbleGen Arrays (obtained from NimbleGen, Madison, Wis.) as indicated in FIG. 15 (e.g. “23 T” or “30T”). In this figure, “cap” refers to the protecting group DMT added during oligonucleotide synthesis and left on to protect the 5′ end of the oligonucleotide. No loop refers to SEQ ID NO:1 (5′-DMT-tttgaggtatacaggtatttgtc-3′), which does not fold on itself as pictured in FIG. 12. For the remaining oligonucleotides, the bases that anneal to form the self complementary loop regions are underlined and in bold; the bases complementary to the “universal” labeling cassette are indicated in italics; the capitalized base is changed to an A in the mutant sequences. “4 loop” refers to a loop structure comprising a 4-bp self complementary region, e.g. SEQ ID NO:2 (5′-DMT-ttttGaggtatacaggtatttgtcacctcattagattac-3′); “6 loop” refers to a loop structure comprising a 4-bp self complementary region, e.g. SEQ ID NO:3 (5′-DMT-ttttGaggtatacaggtatttgtcgtatacctcattagattac-3′); “8 loop” refers to a loop structure comprising a 4-bp self complementary region, e.g. SEQ ID NO:4 (5′-DMT-ttttGaggtatacaggtatttgtcgtatacctcattagattac-3′); “10-loop” refers to a loop structure comprising a 4-bp self complementary region, e.g. SEQ ID NO:5 (5′-DMT-ttttGaggtatacaggtatttgtcctgtatacctcattagattac-3′. “Cleaved no loop, phos” refers to the sequence expected from INVADER assay cleavage of SEQ ID NO:1 and comprises SEQ ID NO:6 (5′-PO4-aggtatacaggtatttgtc-3′); “cleaved 4 loop, phos” refers to the sequence expected from INVADER assay cleavage of SEQ ID NO:2 and comprises SEQ ID NO:7 (5′-PO4-aggtatacaggtatttgtcacctcattagattaccattagattac-3′); “cleaved 6 loop, phos” refers to the sequence expected from INVADER assay cleavage of SEQ ID NO:3 and comprises SEQ ID NO:8 (5′-PO4-aggtatacaggtatttgtcatacctcattagattaccattagattac-3′); “cleaved 8 loop, phos” refers to the sequence expected from INVADER assay cleavage of SEQ ID NO:4 and comprises SEQ ID NO:9 (5′-PO4-aggtatacaggtatttgtcgtatacctcattagattaccattagattac-3′).

Replicate sets of arrays as in FIG. 15 were created. One array was incubated with an 8-mer degenerate or random cassette, SEQ ID NO:10 (5′-cy3-ttttt(n)₈ggcacacgagatttttctcgtgtgcc-3′); one with a 6-mer random cassette, SEQ ID NO:11 (5′-cy3-ttttt(n)₆ggcacacgagatttttctcgtgtgcc-3′); one with a “universal” label complementary to a portion of the looped probes, SEQ ID NO:12 (5′-cy3-tttttgtaatctaatg-3′) as indicated above, and one with a sequence specific cassette SEQ ID NO:13 (5′-cy3-ttttttacctgtatacctggcacacgagatttttctcgtgtgccaggtatacaggtattttgtc-3′). Ligation reactions were carried out according to the following procedure:

| | Sequence | | 6-mer | 8-mer |
|---|---|---|---|---|
| | specific | Universal | random | random |
| Component | cassette | cassette | cassette | cassette |
| 10 × T4 ligase | 1 μl | 1 μl | 1 μl | 1 μl |
| buffer | | | | |
| T4 DNA ligase | 1 μl | 1 μl | 1 μl | 1 μl |
| SEQ ID NO: 13 | 0.5 μl | — | — | — |
| (10 μM) | | | | |
| SEQ ID NO: 12 | — | 0.5 μl | — | — |
| (10 μM) | | | | |
| SEQ ID NO: 10 | — | — | — | 0.5 μl |
| (10 μM) | | | | |
| SEQ ID NO: 11 | — | — | 0.5 μl | — |
| (10 μM) | | | | |
| Water | 7 μl | 7 μl | 7 μl | 7 μl |

Aliquots of 2 μl of the appropriate reaction mixtures were applied to the appropriate zone on a TEFLON template. The chip was affixed to the template and incubated at 30-33° C. for 1 hour. The chip sandwich was disassembled in a room temperature bath of 1% Tween 20, washed once for 5 minutes in 95° C. 0.1% Tween, and then washed three times in water at 95° C., and dried with argon.

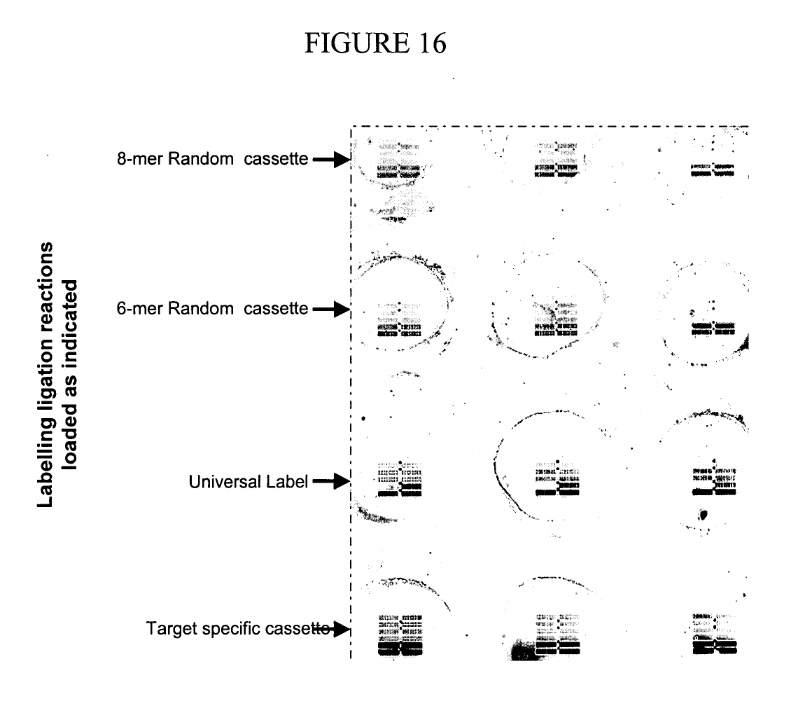

The Cy-3 label was detected with an Alpha Array 7000 (from Alpa Innotech, San Leandro, Calif.) and the results are presented in FIG. 16. The results indicate that label was incorporated with all four cassette types, albeit at a low level with the 8-mer random cassette. In each case, no label was ligated onto the full-length probe molecules (in the top 4 rows of each array) as expected. The four samples at the bottom of each array contained mock cleaved probes designed to serve as substrates for the various ligation reactions. Consistent with the oligonucleotide designs, the target specific product did not hybridize to the “universal” label cassette, since the complement to the “universal” cassette was not comprised in the ASR specific product. The other cassettes, i.e. the two random and the one target specific cassette, hybridized and were ligated to the mock cleaved products. This example indicates that it is possible to use a generic or “universal” approach to label invasive cleavage reaction products on solid surfaces.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A method for detecting the presence of a target nucleic acid in a sample, comprising:
a) providing a microchamber containing a liquid reaction mixture, said liquid reaction mixture comprising:
i) a sample suspected of containing a target nucleic acid, and
ii) detection assay reagents comprising at least one oligonucleotide configured to form a cleavage structure with said target nucleic acid, a cleavage means capable of cleaving a cleavage structure, wherein said detection assay reagents are configured to produce a detectable signal upon cleavage of a cleavage structure;
b) incubating said microchamber under conditions that permit said cleavage reagents to produce a detectable signal; and
c) detecting said detectable signal, thereby detecting said target nucleic acid in said sample.

2. The method of claim 1, wherein said microchamber comprises a capillary tube.

3. The method of claim 1, wherein said detection assay reagents comprise reagents for conducting an invasive cleavage reaction.

4. The method of claim 1, wherein said detection assay reagents comprise a ligase.

5. The method of claim 1, wherein said sample comprises a blood sample.

6. The method of claim 1, wherein said sample is derived from a blood sample.

7. The method of claim 1, wherein said microchamber comprises a coating.

8. The method of claim 7, wherein said coating is selected from the group consisting of C-18, diol, PEG, cyclodextrine, PVA, polystyrene.

9. The method of claim 1, wherein said microchamber comprises a blocking agent.

10. The method of claim 2, wherein said providing comprises:
a) preparing said capillary tube to contain said detection assay reagents in dried form within said capillary tube; and
b) adding said sample suspected of containing a target nucleic acid in liquid form to said capillary tube under conditions such that said liquid reaction mixture is formed.

11. The method of claim 2, wherein said providing comprises:
a) preparing said capillary tube to contain said sample suspected of containing a target nucleic, in dried form within said capillary tube; and
b) adding said detection assay reagents in liquid form to said capillary tube under conditions such that said liquid reaction mixture is formed.

12. A method for detecting the presence of one or more target nucleic acids in a sample, comprising:
a) providing a plurality of microchambers, each microchamber containing a liquid reaction mixture, each said liquid reaction mixture comprising:
i) a sample suspected of containing at least one target nucleic acid, and
ii) detection assay reagents comprising at least one oligonucleotide configured to form a cleavage structure with a target nucleic acid, a cleavage means capable of cleaving a cleavage structure, wherein said detection assay reagents are configured to produce a detectable signal upon cleavage of a cleavage structure;
b) wherein at least two of said plurality of microchambers are configured to detect at least two different target nucleic acids,
c) incubating said plurality of microchambers under conditions that permit said cleavage reagents to produce detectable signal; and
d) detecting said detectable signal, thereby detecting said one or more target nucleic acids in said sample.

13. The method of claim 12, wherein said detection assay reagents comprise reagents for conducting an invasive cleavage reaction.

14. The method of claim 12 wherein said detection assay reagents comprise a ligase.

15. The method of claim 12, wherein said microchamber comprises a capillary tube.

16. The method of claim 15, wherein said providing comprises:
a) preparing said capillary tube to contain said detection assay reagents in dried form within said capillary tube; and
b) adding said sample suspected of containing a target nucleic acid in liquid form to said capillary tube under conditions such that said liquid reaction mixture is formed.

17. The method of claim 15, wherein said providing comprises:
a) preparing said capillary tube to contain said sample suspected of containing a target nucleic, in dried form within said capillary tube; and
b) adding said detection assay reagents in liquid form to said capillary tube under conditions such that said liquid reaction mixture is formed.

18. A kit comprising a plurality of capillary tubes comprising dried detection assay reagents configured to detect target nucleic acids, said dried detection assay reagents comprising reagents for conducting an invasive cleavage reaction.

19. The kit of claim 18, wherein one or more of said capillary tubes having detection assay reagents is configured to a target nucleic acid that at least one other capillary tube in said plurality of capillary tubes is not configured to detect.

20. The kit of claim 18, wherein each of said plurality of capillary tubes comprises detection assay reagents configured to detect a different target nucleic acid.

21. The kit of claim 19, wherein each of said plurality of capillary tubes further comprises detection assay reagents configured to detect the same target nucleic acid.

22. The kit of claim 20, wherein each of said plurality of capillary tubes further comprises detection assay reagents configured to detect the same target nucleic acid.
